(19) United States
(12) Patent Application Publication
(10) Pub. No.: US 2010/0168174 A1
(43) Pub. Date: Jul. 1, 2010

(54) Combination of AMPA Receptor Antagonists and Acetylcholinesterase Inhibitors for the Treatment of Neuropathic Pain

(75) Inventor: Takahisa Hanada, Tsukuba-shi (JP)

Correspondence Address: CLARK & ELBING LLP, 101 FEDERAL STREET, BOSTON, MA, 02110, US

(73) Assignee: Eisai R&D Management Co., Ltd., Tokyo (JP)

(21) Appl. No.: 12/663,550

(22) PCT Filed: Jul. 11, 2008
(86) PCT No.: PCT/JP2008/062976

§ 371 Date: Jan. 4, 2010

Related U.S. Application Data
(60) Provisional application No. 60/929,812, filed on Jul. 13, 2007.

(51) Int. Cl. A61K 31/444; A61P 25/00

(52) U.S. Cl. 514/334

(57) ABSTRACT

The invention provides methods for treating and/or preventing neuropathic pain by administering to patients in need thereof therapeutically effective amounts of AMPA receptor antagonists; and cholinesterase inhibitors and/or anti-neuropathic pain agents. The neuropathic pain may be painful diabetic neuropathy. The invention also provides kits, and pharmaceutical compositions comprising therapeutically effective amounts of AMPA receptor antagonists; and cholinesterase inhibitors and/or anti-neuropathic pain agents. The AMPA receptor antagonists may be, for example, 3-(2-cyanophenyl)-5-(2-pyridyl)-1-phenyl-1,2-dihydropyridin-2-one. The cholinesterase inhibitor may be, for example, 1-benzyl-4-((5,6-dimethoxy-1-indanon)-2-yl)methylpiperidine (donepezil).

### RELATED APPLICATION

This application claims priority under 35 U.S.C. §119 to U.S. Provisional Application No. 60/929,812 filed on Jul. 13, 2007, the disclosures of which are incorporated by reference herein in their entirety.

### FIELD OF THE INVENTION

The invention provides pharmaceutical compositions comprising AMPA receptor antagonists and methods for treating a variety of diseases and disorders using AMPA receptor antagonists. The AMPA receptor antagonists can optionally be used in conjunction with other drugs, such as cholinesterase inhibitors and/or anti-neuropathic pain agents, for treating a variety of diseases and disorders.

### BACKGROUND OF THE INVENTION

Neuropathic pain, caused by various central and peripheral nerve damage or dysfunction, is problematic because of its severity, chronicity and resistance to usual analgesics. Antidepressant (tricyclic antidepressant and duroxetine), antiepileptics (carbamazepine, oxacarbazepine, gabapentin, pregabalin etc.), opioids, and NMDA antagonists are commonly used for the treatment of neuropathic pain but those compounds have a problem in efficacy and tolerability (Finnerup N B. et al., Pain 118 (2005)289-305).

AMPA receptor antagonists include 1,2-dihydropyridine compounds. An exemplary 1,2-dihydropyridine compound is perampanel [3-(2-cyanophenyl)-5-(2-pyridyl)-1-phenyl-1,2-dihydropyridin-2-one], and is described in U.S. Pat. No. 6,949,571. Methods for treating diseases and administering these compounds in conjunction with a cholinesterase inhibitor are described in WO 2006/107859, and WO 2006/107860.

Cholinesterase inhibitors have conventionally been used to treat dementia, such as Alzheimer's Disease and Senile Dementia of the Alzheimer's Type. In particular, donepezil hydrochloride is used extensively as a drug for treating Alzheimer's senile dementia. Donepezil is described in U.S. Pat. No. 4,895,841.

There is a need in the art for treating neuropathic pain using novel pharmaceutical compositions or combinations. The invention is directed to these, as well as other, important goals.

### SUMMARY OF THE INVENTION

The invention provides methods for treatment and/or prophylaxis of neuropathic pain in a patient in need thereof by administering a therapeutically effective amount of at least one AMPA receptor antagonist (e.g., 1,2-dihydropyridine compounds), and, optionally, a therapeutically effective amount of: (i) at least one cholinesterase inhibitor, (ii) at least one anti-neuropathic pain agent, or (iii) at least one cholinesterase inhibitor and at least one anti-neuropathic pain agent. In one embodiment, the AMPA receptor antagonist is 3-(2-cyanophenyl)-5-(2-pyridyl)-1-phenyl-1,2-dihydropyridin-2-one. In one embodiment, the cholinesterase inhibitor is donepezil. The combination of the AMPA receptor antagonist and the cholinesterase inhibitor and/or the anti-neuropathic pain agent unexpectedly produce synergistic effects in the treatment and/or prophylaxis of neuropathic pain.

In other embodiments, the invention provides pharmaceutical compositions comprising a therapeutically effective amount of at least one AMPA receptor antagonist (e.g., 3-(2-cyanophenyl)-5-(2-pyridyl)-1-phenyl-1,2-dihydropyridin-2-one). The invention provides pharmaceutical compositions comprising a therapeutically effective amount of: (i) at least one cholinesterase inhibitor (e.g., donepezil) and (ii) at least one AMPA receptor antagonist (e.g., 3-(2-cyanophenyl)-5-(2-pyridyl)-1-phenyl-1,2-dihydropyridin-2-one). The invention also provides pharmaceutical compositions comprising a therapeutically effective amount of: (i) at least one anti-neuropathic pain agent and (ii) at least one AMPA receptor antagonist (e.g., 3-(2-cyanophenyl)-5-(2-pyridyl)-1-phenyl-1,2-dihydropyridin-2-one). The invention provides pharmaceutical compositions comprising a therapeutically effective amount of: (i) at least one cholinesterase inhibitor (e.g., donepezil); (ii) at least one AMPA receptor antagonist (e.g., 3-(2-cyanophenyl)-5-(2-pyridyl)-1-phenyl-1,2-dihydropyridin-2-one); and (iii) at least one anti-neuropathic pain agent.

The present invention relates to the following:

(1) A pharmaceutical composition comprising:

(A) an AMPA receptor antagonist, a pharmaceutically acceptable salt thereof, a hydrate thereof, or a hydrate of a pharmaceutically acceptable salt thereof;

(B) a cholinesterase inhibitor or a pharmaceutically acceptable salt thereof; an anti-neuropathic pain agent; or a mixture or combination thereof; and

(C) one or more pharmaceutically acceptable carriers.

(2) The pharmaceutical composition of (1), wherein the AMPA receptor antagonist, pharmaceutically acceptable salt thereof, hydrate thereof, or hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is:

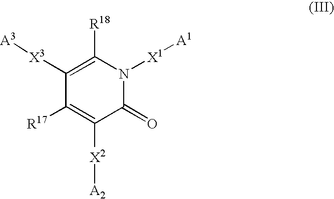

wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

(3) The pharmaceutical composition of (1), wherein the AMPA receptor antagonist, a pharmaceutically acceptable salt thereof, a hydrate thereof, or a hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

(4) The pharmaceutical composition of (1), wherein the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof; and galanthamine or a pharmaceutically acceptable salt thereof.

(5) The pharmaceutical composition of (1), wherein the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818.

(6) The pharmaceutical composition of (1), wherein the composition is used for treating neuropathic pain.

(7) A combination comprising:

(A) an AMPA receptor antagonist, a pharmaceutically acceptable salt thereof, a hydrate thereof, or a hydrate of a pharmaceutically acceptable salt thereof; and

(B) a cholinesterase inhibitor or a pharmaceutically acceptable salt thereof; an anti-neuropathic pain agent; or a mixture or combination thereof.

(8) The combination of (7), wherein the AMPA receptor antagonist, pharmaceutically acceptable salt thereof, hydrate thereof, or hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is:

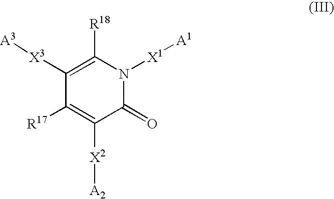

wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)_{P}—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

(9) The combination of (7), wherein the AMPA receptor antagonist, a pharmaceutically acceptable salt thereof, a hydrate thereof, or a hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

(10) The combination of (7), wherein the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof, and galanthamine or a pharmaceutically acceptable salt thereof.

(11) The combination of (7), wherein the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818.

(12) The combination of (7), wherein (A) and (B) are administered separately to a patient or are administered to a patient in the form of a pharmaceutical composition.

(13) The combination of (7), wherein the combination is used for treating neuropathic pain.

(14) Use of compounds (A) and (B) for producing a pharmaceutical composition in the treatment of neuropathic pain, wherein (A) and (B) are:

(A) an AMPA receptor antagonist, a pharmaceutically acceptable salt thereof, a hydrate thereof, or a hydrate of a pharmaceutically acceptable salt thereof; and

(B) a cholinesterase inhibitor or a pharmaceutically acceptable salt thereof; an anti-neuropathic pain agent; or a mixture or combination thereof.

(15) The use of (14), wherein the AMPA receptor antagonist, pharmaceutically acceptable salt thereof, hydrate thereof, or hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is:

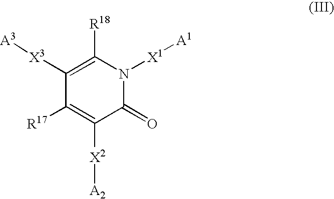

wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

(16) The use of (14), wherein the AMPA receptor antagonist, a pharmaceutically acceptable salt thereof, a hydrate thereof, or a hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

(17) The use of (14), wherein the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof; and galanthamine or a pharmaceutically acceptable salt thereof.

(18) The use of (14), wherein the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818.

(19) The use of (14), wherein (A) and (B) are administered separately to a patient or are administered to a patient in the form of a pharmaceutical composition.

(20) Compounds (A) and (B) for use in the treatment of neuropathic pain, wherein (A) and (B) are:

(A) an AMPA receptor antagonist, a pharmaceutically acceptable salt thereof, a hydrate thereof, or a hydrate of a pharmaceutically acceptable salt thereof; and

(B) a cholinesterase inhibitor or a pharmaceutically acceptable salt thereof; an anti-neuropathic pain agent; or a mixture or combination thereof.

(21) A kit comprising the pharmaceutical composition of any one of (1) to (6) or the combination of any one of (7) to (13).

(22) A method for treating neuropathic pain comprising administering to a patient in need thereof a therapeutically effective amount of the pharmaceutical composition of any one of (1) to (6) or a therapeutically effective amount of the combination of any one of (7) to (13).

### DETAILED DESCRIPTION OF THE INVENTION

“Patient” refers to animals, preferably mammals, more preferably humans. The term “patient” includes men and women; and includes adults, children and neonates. In one embodiment, the patient can be an animal companion, such as a dog or a cat.

“Active ingredient” refers to the AMPA receptor antagonists, cholinesterase inhibitors, anti-neuropathic pain agents, and other compounds described herein that are responsible for treatment and/or prophylaxis of a disease or disorder. The active ingredients may have mechanisms of action that are known or unknown, and the active ingredients may have one or more mechanisms of action. The active ingredient may have an asymmetric carbon depending on the type of substituent and may have a stereoisomer (e.g., a geometric isomer, an enantiomer, a diastereomer or the like). The active ingredient or a stereoisomer thereof may form a pharmaceutically acceptable salt. The active ingredient, a pharmaceutically acceptable salt thereof, a stereoisomer thereof or a pharmaceutically acceptable salt of a stereoisomer thereof may be an anhydride, and may form a solvate. The active ingredient, a pharmaceutically acceptable salt thereof, a stereoisomer thereof, a pharmaceutically acceptable salt of a stereoisomer thereof or a solvate thereof may be crystalline or amorphous. Crystalline polymorphs may exist in the active ingredient, a pharmaceutically acceptable salt thereof, a stereoisomer thereof, a pharmaceutically acceptable salt of a stereoisomer thereof or a solvate thereof, although not limited thereto and any form of crystal may exist alone or in combination, which are within the scope of the present invention.

“Treatment” and “treating” refer to the acquisition of a desired pharmacological effect and/or physiologic effect. These effects are prophylactic in terms of completely or partially preventing a disease and/or symptom(s), and therapeutic in terms of partially or completely curing a disease and/or an adverse event caused by a disease. “Treatment” and “treating” include any treatment of a disease in a patient including, for example: (a) to prevent a disease or symptom(s) in a patient who is suspected of being predisposed to the disease or symptom(s) but not yet diagnosed to be so; (b) to inhibit a symptom(s) of a disease, i.e., to inhibit or delay the progression of the symptom(s); and (c) to alleviate a symptom(s) of a disease, i.e., to reverse or eliminate the symptom(s) of the disease; or to reverse the progress of the symptom(s).

“Administered separately” with reference to the administration of two or more compounds to treat and/or prevent and/or delay the onset of the diseases and disorders described herein includes, for example, the sequential administration of the compounds in any order or the simultaneous administration of the compounds. Simultaneous administration of the compounds means that the compounds are administered to the patient at substantially the same time or at exactly the same time, depending on the mode of administration. The sequential administration of the compounds may occur in any order and may occur with any amount of time elapsing between administration of the compounds. Sequential administration may be based on factors that would influence which of the compounds should be administered first and which should be administered second, and how much time should elapse between administration of the compounds. For example, when two or more compounds are administered separately and sequentially, factors that effect when the compounds are administered to the patient include, for example, (a) the time(s) that provides the best efficacy for the compound being administered, (b) the time(s) that provides the fewest side effects for the compound being administered, (c) the dosage of the compound, (d) the route of administration of the compound, (e) the disease or disorder being treated, (f) the patient being treated, (g) the in vivo relationship of the compounds being administered, and other such factors known in the art. Preferably, the time intervals for sequential administration are chosen so that the effect on the disease or disorder being treated in the combined use of the active ingredients is greater than additive when compared to the effect which would be obtained by use of only one of the active ingredients.

“Combination” refers to the AMPA receptor antagonist and the second active ingredient (e.g., cholinesterase inhibitors or anti-neuropathic pain agents) being administered separately as distinct pharmaceutical compositions or formulations (e.g., a first pharmaceutical composition comprising a AMPA receptor antagonist and a second pharmaceutical composition comprising a cholinesterase inhibitor or anti-neuropathic pain agents). In another embodiment, “combination” refers to the AMPA receptor antagonist, the second active ingredient (e.g., cholinesterase inhibitors) and the third active ingredient (e.g., anti-neuropathic pain agents) being administered separately as distinct pharmaceutical compositions or formulations (e.g., a first pharmaceutical composition comprising an AMPA receptor antagonist, a second pharmaceutical composition comprising a cholinesterase inhibitor and a third pharmaceutical composition comprising an anti-neuropathic pain agent). The pharmaceutical compositions or formulations can have the same or different modes of administration.

“Monotherapy” is a therapy which uses only one active ingredient for treatment and/or prophylaxis of a disease or disorder.

“Combination therapy” is a therapy where two or more active ingredients are administered separately or are administered in the form of a pharmaceutical composition for the treatment and/or prophylaxis of a disease.

“Therapeutically effective amount” refers to the amount of the active ingredient that is necessary for the treatment and/or prophylaxis of a disease. When two or more active ingredients are administered for combination therapy, the term “therapeutically effective amount” refers to the amount of active ingredients that are necessary for treatment and/or prophylaxis of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in combination therapy to achieve a result that would be equal to or greater than the result that the second active ingredient would achieve if it was used for monotherapy); (c) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which in combination therapy provide for treatment and/or prophylaxis of a disease or disorder (e.g., the sub-therapeutic amount of the first active ingredient can be used in combination therapy to achieve a result that would be equal to or greater than the result that the first active ingredient would achieve if it was used for monotherapy; and the sub-therapeutic amount of the second active ingredient can be used in combination therapy to achieve a result that would be equal to or greater than the result that the second active ingredient would achieve if it was used for monotherapy). The same therapeutic/sub-therapeutic amounts can be used when there are three or more active ingredients used in combination therapy. For example, (a) there may be therapeutically effective amounts of all three active ingredients; (b) there may be therapeutically effective amounts of two active ingredients and a sub-therapeutic amount of a third active ingredient; (c) there may be a therapeutically effective amount of one active ingredient and sub-therapeutic amounts of two other active ingredients; or (d) there may be sub-therapeutic amounts of all three active ingredients.

“Kits,” also referred to as “commercial packages,” can include a combination of (i) a first pharmaceutical composition or formulation comprising the AMPA receptor antagonist; (ii) a second pharmaceutical composition or formulation comprising the second active ingredient (e.g., cholinesterase inhibitors or anti-neuropathic pain agents); (iii) instructions for using the pharmaceutical compositions or formulations for treating or preventing or delaying the onset of the disease; and (iv) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. In another embodiment, “kits” can include (i) pharmaceutical composition or formulation comprising both the AMPA receptor antagonist and the second active ingredient (e.g., cholinesterase inhibitors or anti-neuropathic pain agents); (ii) instructions for using the pharmaceutical composition or formulation for treating or preventing or delaying the onset of the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. Moreover, “kits” can include a combination of (i) a first pharmaceutical composition or formulation comprising the AMPA receptor antagonist; (ii) a second pharmaceutical composition or formulation comprising the second active ingredient (e.g., cholinesterase inhibitors); (iii) a third pharmaceutical composition or formulation comprising the third active ingredient (e.g., anti-neuropathic pain agents); (iv) instructions for using the pharmaceutical compositions or formulations for treating or preventing or delaying the onset of the disease; and (v) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. In another embodiment, “kits” can include (i) pharmaceutical composition or formulation comprising all of the AMPA receptor antagonist, the second active ingredient (e.g., cholinesterase inhibitors), and the third active ingredient (e.g., anti-neuropathic pain agents); (ii) instructions for using the pharmaceutical composition or formulation for treating or preventing or delaying the onset of the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months.

“Solvate” is well known in the art. The solvate is preferably a pharmaceutically acceptable solvate. The pharmaceutically acceptable solvate may be either a hydrate or a nonhydrate, but preferably a hydrate. The solvent such as water, alcohol (e.g., methanol, ethanol, n-propanol), dimethylformamide, dimethyl sulfoxide (DMSO) or the like may be used.

“Hydrate” refers to a compound containing a molecule of water of crystallization. The molecule of water of crystallization can be an integer of 1 or more, such as 1 to 10; or can be any fraction greater than 0 or a fraction of an integer from 1 to 10. For example, the hydrate may be represented as compound.¼H₂O; compound.½H₂O; compound.¾H₂O; compound.2H₂O; compound.5½H₂O; compound.6H₂O; and the like. The “compound” can be any described herein, such as 3-(2-cyanophenyl)-5-(2-pyridyl)-1-phenyl-1,2-dihydropyridin-2-one.

“Pharmaceutically acceptable salts” are well known in the art and include those of inorganic acids, such as hydrochloride, sulfate, hydrobromide and phosphate; and those of organic acids, such as formate, tartrate, acetate, trifluoroacetate, methanesulfonate, benzenesulfonate and toluenesulfonate. When certain substituents are selected, the compounds of the invention can form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylenediamine. One skilled in the art will recognize that the compounds of the invention can be made in the form of any other pharmaceutically acceptable salt.

“Neuropathic pain” refers to a complex, chronic pain state that usually is accompanied by tissue injury. With neuropathic pain, the nerve fibers themselves may be damaged, dysfunctional or injured. These damaged nerve fibers send incorrect signals to other pain centers. The impact of nerve fiber injury includes a change in nerve function both at the site of injury and areas around the injury. Symptoms of neuropathic pain may include shooting and burning pain and/or tingling and numbness. The neuropathic pain can be any neuropathic pain disease or disorder known in the art. One example of neuropathic pain is called phantom limb syndrome. This occurs when an arm or a leg has been removed because of illness or injury, but the brain still gets pain messages from the nerves that originally carried impulses from the missing limb. These nerves now misfire and cause pain. Neuropathic pain often seems to have no obvious cause; but, some exemplary causes of neuropathic pain include: alcoholism; amputation; back, leg, and hip problems; chemotherapy; diabetes; facial nerve problems; HIV infection; AIDS; multiple sclerosis; shingles; spine surgery, and the like.

Exemplary neuropathic pain includes 1) pain-induced by traumatic mononeuropathies, including entrapment neurpathies, painful scars, partial or complete transsection, post-tharacotomy, causalgia and stump pain; 2) pain-induced by other mononeuropathies and multiple mononeuropathies, including diabetic mononeuropathy, neuralgic amyotrophy, diabetic amyotrophy, malignant nerve/plexus invasion, postherpetic neuropathic pain, radiation plexopathy, trigeminal neuropathic pain, connective tissue disease and glossopharyngeal neuropathic pain; 3) pain-induced by polyneuropathies including (a) caused by metabolic/nutritional abnormality, including diabetic, alcoholic, pellagra, amyroid, beriberi, Strachan's (Jamaican neuropathy), Cuban neuropathy, Tanzanian neuropathy and burning feet syndrome, (b) caused by one or more drugs including isoniazid cisplatin, vincristine, nitrofurantoin and disulfuram, (c) caused by one or more toxic substances including thallium, arsenic and clioquinol, (d) hereditary neuropathies, including Fabry's disease and dominantly inherited sensory neuropathy, (e) caused by malignancy including myeloma and carcinomatous, (f) others including acute idiopathic polyneuropathy (Guillain-Barré) and idiopathic neuropathy; 4) central pain induced by vascular lesions in the brain and spinal cord, traumatic spinal cord injury, syringomyelia and syringobulbia, tumours, abscesses, demyelination and phantom pain.

The diagnosis of neuropathic pain is carried out based on history and finding from physical examination of the patients.

In one embodiment, the AMPA receptor antagonist used in the methods and compositions described herein may be any known in the art. Exemplary AMPA receptor antagonists, all of which are active ingredients, include 1,2-dihydropyridine compounds, quinoxalinedione aminoalkylphosphonates, and the like.

In other embodiments, the AMPA receptor antagonist may be becampanel, EGIS 8332 (7-acetyl-5-(4-aminophenyl)-8,9-dihydro-8-methyl-7,3-1,3-dioxolo[4,5-h][2,3]benzodiazepine-8-carbonitrile); GYKI 47261 (4-(7-chloro-2-methyl-4H-3,10,10a-triaza-benzo[f]azulen-9-yl)phenylamine)); irampanel (N,N-dimethyl-2-[2-(3-phenyl-1,2,4-oxadiazol-5-yl)phenoxy]lethanamine); KRP 199 ((7-[4-[[[[4-carboxyphenyl)-amino]carbonyl]oxy]methyl]-1H-imidazol-1-yl]-3,4-dihydro-3-oxo-6-(trifluoromethyl)-2-quinoxalinecarboxylic acid); NS 1209 (2-[[[5-[4-[(dimethylamino)-sulfonyl]phenyl]-1,2,6,7,8,9-hexahydro-8-methyl-2-oxo-3H-pyrrolo[3,2-h]isoquinolin-3-ylidene]amino]oxy]-4-hydroxybutanoic acid monosodium salt; topiramate (TOPAMAX®); talampanel (LY-300164, (R)-7-acetyl-5-(4-aminophenyl)-8,9-dihydro-8 methyl-7H-1,3-dioxolo[4,5-h][2,3]benzo-diazepine; YM9OK (6-imidazol-1-yl-7-nitro-1,4-dihydro-quinoxaline-2,3-dione); S-34730 (7-chloro-6-sulfamoyl-2-(1H)-quinolinone-3-phosphonic acid); Zonampanel (YM-872; (7-imidazol-1-yl-6-nitro-2,3-dioxo-3,4-dihydro-2H-quinoxalin-1-yl)-acetic acid); GYM 52466 (4-(8-methyl-9H-1,3-dioxa-6,7-diaza-cyclohepta[f]inden-5-yl)-phenylamine); ZK 200775 (MPQX, (7-morpholin-4-yl-2,3-dioxo-6-trifluoromethyl-3,4-dihydro-2H-quinoxalin-1-ylmethyl)-phosphonic acid); CP-465022 (3-(2-chlorophenyl)-2-[2-(6-diethylaminomethyl-pyridin-2-yl)-vinyl]-6-fluoro-3H-quinazolin-4-one); SYM-2189 (4-(4-amino-phenyl)-6-methoxy-1-methyl-1H-phthalazine-2-carboxylic acid propylamide); SYM-2206 (8-(4-amino-phenyl)-5-methyl-5H-[1,3]dioxolo[4,5-g]phthalazine-6-carboxylic acid propylamide); RPR-117824 ((4-oxo-2-phosphono-5,10-dihydro-4H-imidazo[1,2-a]indeno[1,2-e]pyrazin-9-yl)-acetic acid); or LY-293558 (6-[2-(1H-tetrazol-5-yl)-ethyl]-decahydro-isoquinoline-3-carboxylic acid).

In other embodiment, the AMPA receptor antagonist is a 1,2-dihydropyridine compound. The 1,2-dihydropyridine compound used in the methods and compositions described herein may be any known in the art. The term “1,2-dihydropyridine compound” includes 1,2-dihydropyridine compounds, pharmaceutically acceptable salts of 1,2-dihydropyridine compounds, stereoisomers of 1,2-dihydropyridine compounds, pharmaceutically acceptable salts of stereoisomers of 1,2-dihydropyridine compounds, hydrates of 1,2-dihydropyridine compounds, hydrates of pharmaceutically acceptable salts of 1,2-dihydropyridine compounds, stereoisomers of hydrates of 1,2-dihydropyridine compounds, and stereoisomer of hydrates of pharmaceutically acceptable salts of 1,2-dihydropyridine compounds.

The 1,2-dihydropyridine compound used in the methods and compositions described herein may be a compound of Formula (I):

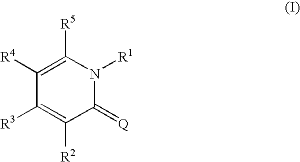

wherein

Q is NH, O or S;

R¹, R², R³, R⁴ and R⁵ are each independently hydrogen, halogen, C₁₋₆ alkyl, or —X-A;

X is a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)_{P}—, —S(O)_{q}—CH₂—, —CH₂—O—, —O—CH₂—, —N(R¹³)—CO—N(R¹⁴)— or —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A is an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring; provided that 3 groups among R¹, R², R³, R⁴ and R⁵ are —X-A; and that the residual 2 groups among R¹, R², R³, R⁴ and R⁵ are independently hydrogen, halogen, or C₁₋₆ alkyl.

In one embodiment, the following compounds are excluded from the scope of the compound of Formula (I): (1) when Q is 0; R¹ and R⁵ are hydrogen; and R², R³ and R⁴ are phenyl; (2) when Q is O; R¹ and R⁴ are hydrogen; and R², R³ and R⁵ are phenyl; and (3) when Q is O; R¹ and R² are hydrogen; and R³, R⁴ and R⁵ are phenyl.

In another embodiment, the 1,2-dihydropyridine compound used in the methods and compositions described herein is a compound of Formula (II):

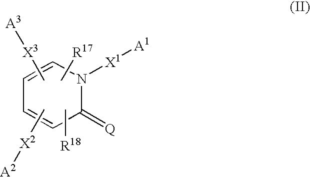

wherein

Q is NH, O or S;

X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);

R⁶, R⁷, R⁸, R⁹, R¹⁰, R¹¹, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and

R¹⁷ and R¹⁸ are each independently hydrogen, halogen, or C₁₋₆ alkyl.

In another embodiment, the invention provides the compound of Formula (II) wherein X¹, X² and X³ are each independently a single bond, an optionally substituted C₁₋₆ alkylene, an optionally substituted C₂₋₆ alkenylene, or an optionally substituted C₂₋₆ alkynylene. The substituents may be one or more of —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— and —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring.

The substituents for the 1,2-dihydropyridine compounds of the invention may be one or more of hydroxy; halogen; nitrile; nitro; C₁₋₆ alkyl; C₂₋₆ alkenyl; C₂₋₆ alkynyl [wherein the alkyl, alkenyl, and alkynyl can independently and optionally be substituted with one or more groups selected from hydroxy, nitrile, halogen, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, alkyl-N—C₂₋₆ alkenylamino, N—C₁₋₆ alkyl-N—C₂₋₆ alkynylamino, N—C₂₋₆ alkenyl-N—C₂₋₆ alkynylamino, aralkyloxy, TBDMS oxy, C₁₋₆ alkylsulfonylamino, C₁₋₆ alkylcarbonyloxy, C₂₋₆ alkenylcarbonyloxy, C₂₋₆ alkynylcarbonyloxy, N—C₁₋₆ alkylcarbamoyl, N—C₂₋₆ alkenylcarbamoyl, and N—C₁₋₆ alkynylcarbamoyl]; C₁₋₆ alkoxy; C₂₋₆ alkenyloxy; C₂₋₆ alkynyloxy [wherein the alkoxy, alkenyloxy, and alkynyloxy may independently and optionally be substituted with one or more groups selected from C₁₋₆ alkylamino, aralkyloxy, and hydroxy]; C₁₋₆ alkylthio; C₂₋₆ alkenylthio; C₂₋₆ alkynylthio [wherein the alkylthio, alkenylthio, and alkynylthio may independently and optionally be substituted with one or more groups selected from hydroxy, nitrile, halogen, C₁₋₆ alkylamino, aralkyloxy, TBDMS oxy, C₁₋₆ alkylsulfonylamino, C₁₋₆ alkylcarbonyloxy, and C₁₋₆ alkylcarbamoyl]; optionally substituted carbonyl [which may be substituted with C₁₋₆ alkoxy, amino, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, N—C₁₋₆ alkyl-N—C₂₋₆ alkenylamino, N—C₁₋₆ alkyl-N—C₂₋₆ alkynylamino and N—C₂₋₆ alkenyl-N—C₂₋₆ alkynylamino]; an optionally substituted amino [which may be substituted with one or two groups selected from C₁₋₆ alkyl, C₂₋₆ alkenyl, C₂₋₆ alkynyl, C₁₋₆ alkylsulfonyl, C₂₋₆ alkenylsulfonyl, C₂₋₆ alkynylsulfonyl, C₁₋₆ alkylcarbonyl, C₂₋₆ alkenylcarbonyl and C₂₋₆ alkynylcarbonyl]; C₁₋₆ alkylsulfonyl; C₂₋₆ alkenylsulfonyl; C₂₋₆ alkynylsulfonyl; C₁₋₆ alkylsulfinyl; C₂₋₆ alkenylsulfinyl; C₂₋₆ alkynylsulfinyl; formyl; optionally substituted C₂₋₈ cycloalkyl; an optionally substituted C₃₋₈ cycloalkenyl [where the cycloalkyl group and/or the cycloalkenyl group may independently and optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; a 5- to 14-membered non-aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; C₆₋₁₄ aromatic hydrocarbocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; and a 5- to 14-membered aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl].

In another embodiment, the invention provides compounds of Formula (II) wherein A¹, A² and A³ are each independently an optionally substituted C₃₋₈ cycloalkyl, an optionally substituted C₃₋₈ cycloalkenyl or an optionally substituted 5- to 14-membered non-aromatic hetero ring. In another embodiment, the invention provides the compound of Formula (II) wherein A¹, A² and A³ are each independently an optionally substituted C₆₋₁₄ aromatic hydrocarbon ring or an optionally substituted 5- to 14-membered aromatic hetero ring. In another embodiment, the invention provides the compound of Formula (II) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl or morpholyl; any of which may optionally have substituents. In another embodiment, the invention provides the compound of Formula (II) wherein A¹, A² and A³ are each independently selected from:

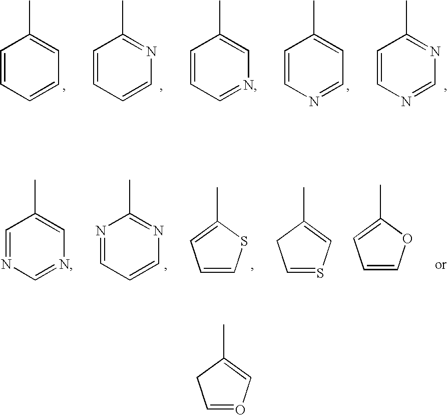

each of which may optionally be substituted. In another embodiment, the invention provides the compound of Formula (II) wherein A¹, A² and A³ are each independently substituted with hydroxyl, halogen, amino, or nitrile. In another embodiment, the invention provides the compound of Formula (II) wherein A¹, A² and A³ are each independently hydroxyl, halogen, amino, nitrile, or nitro. In another embodiment, the invention provides the compound of Formula (II) wherein Q is oxygen.

In another embodiment, the invention provides the compounds of Formula (I) or (II) wherein X² and X³ are each independently a single bond, —CH₂—, —CH(OH)—, —CH₂—CH₂—, —CH═CH—, —C≡C—, —O— or —CO—. In another embodiment, the invention provides the compounds of Formula (I) or (II) wherein X² and X³ are each a single bond. In another embodiment, the invention provides the compounds of Formula (I) or (II) wherein R¹⁷ and R¹⁸ are each independently hydrogen, fluorine, chlorine, bromine, iodine, methyl, ethyl, n-propyl, or iso-propyl. In another embodiment, the invention provides the compounds of Formula (I) or (II) wherein R¹⁷ and R¹⁸ are each hydrogen.

With respect to the 1,2-dihydropyridine compounds of the invention, the halogen atom indicates fluorine, chlorine, bromine, iodine and the like, and the preferable atoms include fluorine, chlorine and bromine.

With respect to the 1,2-dihydropyridine compounds of the invention, the C₁₋₆ alkyl indicates an alkyl having 1 to 6 carbons, and examples include linear chain or branched chain alkyl groups such as methyl, ethyl, n-propyl, iso-propyl, n-butyl, iso-butyl, sec-butyl, tert-butyl, n-pentyl, 1,1-dimethylpropyl, 1,2-dimethylpropyl, 2,2-dimethylpropyl, 1-ethylpropyl, 2-ethylpropyl, n-hexyl, 1-methyl-2-ethylpropyl, 1-ethyl-2-methylpropyl, 1,1,2-trimethylpropyl, 1-propylpropyl, 1-methylbutyl, 2-methylbutyl, 1,1-dimethylbutyl, 1,2-dimethylbutyl, 2,2-dimethylbutyl, 1,3-dimethylbutyl, 2,3-dimethylbutyl, 2-ethylbutyl, 2-methylpentyl, 3-methylpentyl, and the like.

With respect to the 1,2-dihydropyridine compounds of the invention, the C₂₋₆ alkenyl indicates an alkenyl group having 2 to 6 carbons, and examples include vinyl, allyl, 1-propenyl, 2-propenyl, iso-propenyl, 2-methyl-1-propenyl, 3-methyl-1-propenyl, 2-methyl-2-propenyl, 3-methyl-2-propenyl, 1-butenyl, 2-butenyl, 3-butenyl, 1-pentenyl, 1-hexenyl, 1,3-hexadienyl, 1,6-hexadienyl, and the like.

With respect to the 1,2-dihydropyridine compounds of the invention, the C₂₋₆ alkynyl indicates an alkynyl group having 2 to 6 carbons, and examples include ethynyl, 1-propynyl, 2-propynyl, 1-butynyl, 2-butynyl, 3-butynyl, 3-methyl-1-propynyl, 1-ethynyl-2-propynyl, 2-methyl-3-propynyl, 1-pentynyl, 1-hexynyl, 1,3-hexadiynyl, 1,6-hexadiynyl, and the like.

With respect to the 1,2-dihydropyridine compounds of the invention, the C₁₋₆ alkoxy indicates an alkoxy group having 1 to 6 carbons, and examples include methoxy, ethoxy, n-propoxy, iso-propoxy, sec-propoxy, n-butoxy, iso-butoxy, sec-butoxy, tert-butoxy, n-pentyloxy, iso-pentyloxy, sec-pentyloxy, n-hexoxy, iso-hexoxy, 1,1-dimethylpropoxy, 1,2-dimethylpropoxy, 2,2-dimethylpropoxy, 2-ethylpropoxy, 1-methyl-2-ethylpropoxy, 1-ethyl-2-methylpropoxy, 1,1,2-trimethylpropoxy, 1,1,2-trimethylpropoxy, 1,1-dimethylbutoxy, 1,2-dimethylbutoxy, 2,2-dimethylbutoxy, 2,3-dimethylbutoxy, 1,3-dimethylbutoxy, 2-ethylbutoxy, 1,3-dimethylbutoxy, 2-methylpentoxy, 3-methylpentoxy, hexyloxy, and the like.

With respect to the 1,2-dihydropyridine compounds of the invention, the C₂₋₆ alkynyloxy indicates an alkynyloxy group having 2 to 6 carbon atoms, and examples include ethynyloxy, 1-propynyloxy, 2-propynyloxy, 1-butynyloxy, 2-butynyloxy, 3-butynyloxy, 1-methyl-2-propynyloxy, 1-ethyl-2-propynyloxy, 1-ethynyl-2-propynyloxy, 1-pentynyloxy, 1-hexynyloxy, 1,3-hexadiynyloxy, 1,6-hexadiynyloxy, and the like.

With respect to the 1,2-dihydropyridine compounds of the invention, the C₂₋₆ alkenyloxy indicates an alkenyloxy group having 2 to 6 carbons, and examples include vinyloxy, 2-propenyloxy, 1-propenyloxy, 2-propenyloxy, iso-propenyloxy, 2-methyl-1-propenyloxy, 3-methyl-1-propenyloxy, 2-methyl-2-propenyloxy, 3-methyl-2-propenyloxy, 1-butenyloxy, 2-butenyloxy, 3-butenyloxy, 1-pentenyloxy, 1-hexenyloxy, 1,3-hexadienyloxy, 1,6-hexadienyloxy, and the like.

With respect to the 1,2-dihydropyridine compounds of the invention, the C₃₋₈ cycloalkyl indicates a cycloalkyl group composed of 3 to 8 carbon atoms, and examples include cyclopropyl, cyclobutyl, cyclopentyl, cyclohexyl, cycloheptyl, cyclooctyl, and the like.

With respect to the 1,2-dihydropyridine compounds of the invention, the C₃₋₈ cycloalkenyl indicates a cycloalkenyl group composed of 3 to 8 carbon atoms, and examples include cyclopropen-1-yl, cyclopropen-3-yl, cyclobuten-1-yl, cyclobuten-3-yl, 1,3-cyclobutadien-1-yl, cyclopenten-1-yl, cyclopenten-3-yl, cyclopenten-4-yl, 1,3-cyclopentadien-1-yl, 1,3-cyclopentadien-2-yl, 1,3-cyclopentadien-5-yl, cyclohexen-1-yl, cyclohexen-3-yl, cyclohexen-4-yl, 1,3-cyclohexadien-1-yl, 1,3-cyclohexadien-2-yl, 1,3-cyclohexadien-5-yl, 1,4-cyclohexadien-3-yl, 1,4-cyclohexadien-1-yl, cyclohepten-1-yl, cyclohepten-3-yl, cyclohepten-4-yl, cyclohepten-5-yl, 1,3-cyclohepten-2-yl, 1,3-cyclohepten-1-yl, 1,3-cycloheptadien-5-yl, 1,3-cycloheptadien-6-yl, 1,4-cycloheptadien-3-yl, 1,4-cycloheptadien-2-yl, 1,4-cycloheptadien-1-yl, 1,4-cycloheptadien-6-yl, 1,3,5-cycloheptatrien-3-yl, 1,3,5-cycloheptatrien-2-yl, 1,3,5-cycloheptatrien-1-yl, 1,3,5-cycloheptatrien-7-yl, cycloocten-1-yl, cycloocten-3-yl, cycloocten-4-yl, cycloocten-5-yl, 1,3-cyclooctadien-2-yl, 1,3-cyclooctadien-1-yl, 1,3-cyclooctadien-5-yl, 1,3-cyclooctadien-6-yl, 1,4-cyclooctadien-3-yl, 1,4-cyclooctadien-2-yl, 1,4-cyclooctadien-1-yl, 1,4-cyclooctadien-6-yl, 1,4-cyclooctadien-7-yl, 1,5-cyclooctadien-3-yl, 1,5-cyclooctadien-2-yl, 1,3,5-cyclooctatrien-3-yl, 1,3,5-cyclooctatrien-2-yl, 1,3,5-cyclooctatrien-1-yl, 1,3,5-cyclooctatrien-7-yl, 1,3,6-cyclooctatrien-2-yl, 1,3,6-cyclooctatrien-1-yl, 1,3,6-cyclooctatrien-5-yl, 1,3,6-cyclooctatrien-6-yl group, and the like.

With respect to the 1,2-dihydropyridine compounds of the invention, the 5- to 14-membered non-aromatic heterocyclic ring means a mono-cyclic, di-cyclic, or tri-cyclic 5- to 14-membered non-aromatic heterocyclic ring which contains one or more hetero atoms selected from nitrogen, sulfur, and oxygen. Specific examples include pyrrolidinyl, pyrrolyl, piperidinyl, piperazinyl, imidazolyl, pyrazolidyl, imidazolidyl, morpholyl, tetrahydrofuryl, tetrahydropyranyl, pyrrolinyl, dihydrofuryl, dihydropyranyl, imidazolinyl, oxazolinyl, and the like. Further, a group derived from a pyridone ring and a non-aromatic condensed ring (for example, a group derived from a phthalimide ring, a succinimide ring, and the like) are also included in the non-aromatic heterocyclic ring.

With respect to the 1,2-dihydropyridine compounds of the invention, the C₆₋₁₄ aromatic hydrocarbocyclic ring and the aryl mean an aromatic hydrocarbocyclic ring which is composed of 6 to 14 carbon atoms, a mono-cyclic ring, and a condensed di-cyclic, tri-cyclic and the like. Specific examples include phenyl, indenyl, 1-naphthyl, 2-naphthyl, azulenyl, heptalenyl, biphenyl, indathenyl, acenaphthyl, fluorenyl, phenalenyl, phenanthrenyl, anthracenyl, cyclopentacyclooctenyl, benzocyclooctenyl and the like.

With respect to the 1,2-dihydropyridine compounds of the invention, the 5- to 14-membered aromatic heterocyclic ring and the heteroaryl ring mean mono-cyclic, di-cyclic, or tri-cyclic 5- to 14-membered aromatic heterocyclic ring which contain one or more hetero atoms selected from nitrogen, sulfur, and oxygen. Specific examples include (1) aromatic heterocyclic rings containing nitrogen such as pyrrolyl, pyridyl, pyridazinyl, pyrimidinyl, pyrazinyl, triazolyl, tetrazolyl, benzotriazolyl, pyrazolyl, imidazolyl, benzimidazolyl, indolyl, iso-indolyl, indolizinyl, prenyl, indazolyl, quinolyl, iso-quinolyl, quinoliziyl, phthalazyl, naphthylidinyl, quinoxalyl, quinazolinyl, cynnolinyl, pteridinyl, imidazotriazinyl, pyrazinopyridazinyl, acridinyl, phenanthridinyl, carbazolyl, carbazolinyl, perimidinyl, phenanthrolinyl, phenacinyl, imidazopyridinyl, imidazopyrimidinyl, pyrazolopyridinyl, or pyrazolopyridinyl; (2) aromatic heterocyclic rings containing sulfur such as thienyl or benzothienyl; (3) aromatic heterocyclic rings containing oxygen such as furyl, pyranyl, cyclopentapyranyl, benzofuryl or iso-benzofuryl; and (4) aromatic heterocyclic rings containing 2 or more different hetero atoms such as thiazolyl, iso-thiazolyl, benzothiazolyl, benzthiadiazolyl, phenothiazinyl, isoxazolyl, furazanyl, phenoxazinyl, oxazolyl, isoxazoyl, benzoxazolyl, oxadiazolyl, pyrazoloxadiazolyl, imidazothiazolyl, thienofuranyl, furopyrrolyl or pyridoxadinyl.

In another embodiment, the 1,2-dihydropyridine compound used in the methods and compositions described herein is a compound of Formula (III):

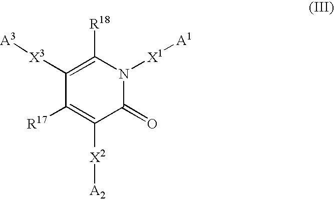

wherein X¹, X², X³, A¹, A², A³, R¹⁷ and R¹⁸ have the same meanings as defined in the above compound of Formula (II).

In another embodiment, the invention provides the compounds of Formula (III) wherein A¹, A² and A³ are each independently an optionally substituted C₆₋₁₄ aromatic hydrocarbon ring or 5- to 14-membered aromatic hetero ring. In another embodiment, the invention provides the compounds of Formula (III) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl, or morpholyl; wherein each may optionally be substituted. In another embodiment, the invention provides the compounds of Formula (III) wherein A¹, A² and A³ are each independently selected from:

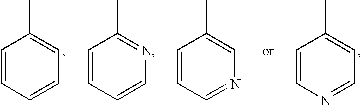

each of which may optionally be substituted. In another embodiment, the invention provides the compounds of Formula (III) wherein the bonding site of the substituent at A¹, A² and A³ are in the α-position of the carbon atom bonding to the group X¹, X² and X³, respectively. In another embodiment, the invention provides the compounds of Formula (III) wherein X¹, X² and X³ are single bonds. In another embodiment, the invention provides the compounds of Formula (III) wherein R¹⁷ and R¹⁸ are hydrogen.

In one embodiment, the 1,2-dihydropyridine compound used in the methods and compositions described herein is preferably Compound A:

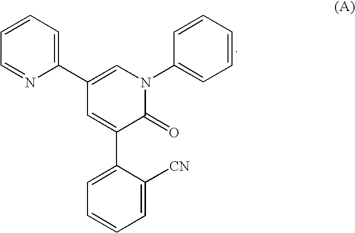

The IUPAC name for Compound A is 2-(2-oxo-1-phenyl-5-pyridin-2-yl-1,2-dihydropyridin-3-yl)benzonitrile. Compound A may also be referred to as 3-(2-cyanophenyl)-5-(2-pyridyl)-1-phenyl-1,2-dihydropyridin-2-one. Compound A is also known as perampanel.

Throughout the specification, the terms “Compound A,” “2-(2-oxo-1-phenyl-5-pyridin-2-yl-1,2-dihydropyridin-3-yl)benzonitrile,” “3-(2-cyanophenyl)-5-(2-pyridyl)-1-phenyl-1,2-dihydropyridin-2-one,” and “perampanel” are intended to include pharmaceutically acceptable salts thereof, stereoisomers thereof, pharmaceutically acceptable salts of stereoisomers thereof, hydrates thereof, hydrates of pharmaceutically acceptable salts thereof, stereoisomers of hydrates

thereof, and stereoisomer of hydrates of pharmaceutically acceptable salts thereof. In another embodiment, the terms “Compound A,” “2-(2-oxo-1-phenyl-5-pyridin-2-yl-1,2-dihydropyridin-3-yl)benzonitrile,” “3-(2-cyanophenyl)-5-(2-pyridyl)-1-phenyl-1,2-dihydropyridin-2-one,” and “perampanel” are intended to include pharmaceutically acceptable salts thereof, hydrates thereof, and hydrates of pharmaceutically acceptable salts thereof.

In other embodiments, the 1,2-dihydropyridine compounds that are useful in the methods and compositions of the invention are 3-(2-cyanophenyl)-5-(2-methylsulfonylaminophenyl)-1-phenyl-1,2-dihydropyridin-2-one; 3-(2-chloro-3-pyridyl)-5-(2-pyridyl)-1-phenyl-1,2-dihydropyridin-2-one; 3-(2-cyanophenyl)-5-(2-pyridyl)-1-phenyl-1,2-dihydropyridin-2-one; 3-(2-cyanophenyl)-5-(2-pyridyl)-1-(3-nitrophenyl)-1,2-dihydropyridin-2-one; 3-(2-cyanophenyl)-5-(2-pyridyl)-1-(3-aminophenyl)-1,2-dihydropyridin-2-one; 3-(2-cyanophenyl)-5-(2-pyridyl)-1-(3-methylsulfonylaminophenyl)-1,2-dihydropyridin-2-one; 3-(2-cyanophenyl)-5-(2-pyridyl)-1-(3-methylaminophenyl)-1,2-dihydropyridin-2-one; 3-(2-cyanophenyl)-5-(2-pyridyl)-1-(3-dimethylaminophenyl)-1,2-dihydropyridin-2-one; 3-(2-cyanophenyl)-5-(2-pyridyl)-1-[3-(5-methoxymethyl-2-oxazolidinon-3-yl)-phenyl]-1,2-dihydropyridin-2-one; 3-(2-cyanophenyl)-5-(2-pyridyl)-1-(3-methoxycarbonylphenyl)-1,2-dihydropyridin-2-one; 3-(2-cyanophenyl)-5-(2-pyridyl)-1-(3-methylaminocarbonylphenyl)-1,2-dihydropyridin-2-one; 3-(2-cyano-3-pyridyl)-5-(2-pyridyl)-1-phenyl-1,2-dihydropyridin-2-one; 3-(2-chlorophenyl)-5-(2-pyridyl)-1-(4-hydroxyphenyl)-1,2-dihydropyridin-2-one; 3-(2-chlorophenyl)-5-(2-pyridyl)-1-(4-dimethylaminoethoxyphenyl)-1,2-dihydropyridin-2-one; 3-(2-cyanophenyl)-5-(2-pyridyl)-1-(3-formylphenyl)-1,2-dihydropyridin-2-one; 3-(2-cyanophenyl)-5-(2-pyridyl)-1-(3-hydroxymethylphenyl)-1,2-dihydropyridin-2-one; 3-(2-cyanophenyl)-5-(2-pyridyl)-1-(3-cyanomethylphenyl)-1,2-dihydropyridine-2-one; 3-(2-cyanophenyl)-5-(2-pyridyl)-1-(3-acetylaminomethylphenyl)-1,2-dihydropyridin-2-one; 3-(2-cyanophenyl)-5-(2-pyridyl)-1-(3-methylsulfonylaminomethylphenyl)-1,2-dihydropyridin-2-one; 3-(2-cyanophenyl)-5-(2-pyridyl)-1-(3-acetoxymethylphenyl)-1,2-dihydropyridin-2-one; 3-(2-cyanophenyl)-5-(2-pyridyl)-1-(4-methylthiophenyl)-1,2-dihydropyridin-2-one; 3-(2-cyanophenyl)-5-(2-pyridyl)-1-(4-methylsulfonylpheny-1)-1,2-dihydropyridin-2-one; 3-(2-cyanophenyl)-5-(2-formylthiophen-3-yl)-1-phenyl-1,2-dihydropyridin-2-one; 3-(2-cyanophenyl)-5-(2-diethylaminomethylthiophen-3-yl)-1-phenyl-1,2-dihydropyridin-2-one; 3-(2-cyanophenyl)-5-(2-hydroxymethylthiophen-3-yl)-1-phenyl-1,2-dihydropyridine-2-one; 3-(2-cyanophenyl)-5-(2-pyridyl)-1-benzyl-1,2-dihydropyridin-2-one; 3-(2-cyanophenyl)-5-(2-pyridyl)-1-(3-pyridyl)-1,2-dihydropyridin-2-one; 3-(2-cyanophenyl)-5-phenyl-(2-pyridyl)-1,2-dihydropyridin-2-one; 3-(2-cyanophenyl)-1,5-diphenyl-1,2-dihydropyridin-2-one; 3-(2-cyanophenyl)-5-(2-methoxyphenyl)-1-phenyl-1,2-dihydropyridin-2-one; 3-(2-cyanophenyl)-5-(3,4-dimethoxyphenyl)-1-phenyl-1,2-dihydropyridin-2-one; 3-(2-cyanophenyl)-5-(thiophen-3-yl)-1-phenyl-1,2-dihydropyridin-2-one; 3-(2-cyanophenyl)-5-(2-fluorophenyl)-1-phenyl-1,2-dihydropyridin-2-one; 3-(2-cyanophenyl)-5-(thiophen-2-yl)-1-phenyl-1,2-dihydropyridin-2-one; 3-(2-cyanophenyl)-5-(3-furfuryl)-1-phenyl-1,2-dihydropyridin-2-one; 3-(2-cyanophenyl)-5-(2-furfuryl)-1-phenyl-1,2-dihydropyridin-2-one; 3-(2-chlorophenyl)-5-(2-pyridyl)-1-phenyl-1,2-dihydropyridin-2-one; 3-(2-methoxycarbonylphenyl)-5-(2-pyridyl)-1-phenyl-1,2-dihydropyridin-2-one; 3-phenyl-5-(2-pyridyl)-1-phenyl-1,2-dihydropyridin-2-one; 3-(2-fluorophenyl)-5-(2-pyridyl)-1-phenyl-1,2-dihydropyridin-2-one; 3-(2-chlorophenyl)-5-(2-pyridyl)-1-(3-methoxyphenyl)-1,2-dihydropyridin-2-one; 3-(2-fluoro-3-pyridyl)-5-(2-pyridyl)-1-phenyl-1,2-dihydropyridin-2-one; 3-(4-methoxy-3-pyridyl)-5-(2-pyridyl)-1-phenyl-1,2-dihydropyridin-2-one; 3-(2-fluoro-3-pyridyl)-5-(2-pyridyl)-1-(3-methoxyphenyl)-1,2-dihydropyridin-2-one; 3-(2-fluoro-3-pyridyl)-5-(2-pyridyl)-1-(3-fluorophenyl)-1,2-dihydropyridin-2-one; 3-(2-cyanophenyl)-5-(2-pyridyl)-1-(4-fluorophenyl)-1,2-dihydropyridin-2-one; 3-(2-cyanophenyl)-5-(2-pyridyl)-1-(3-fluorophenyl)-1,2-dihydropyridin-2-one; 3-(2-cyanophenyl)-5-(2-pyridyl)-1-(4-methoxyphenyl)-1,2-dihydropyridin-2-one; 3-(2-cyanophenyl)-5-(2-pyridyl)-1-(3-methoxy-phenyl)-1,2-dihydropyridin-2-one; 3-phenyl-5-(2-pyridyl)-1-(3-fluorophenyl-)-1,2-dihydropyridin-2-one; 3-(2-chlorophenyl)-5-(2-pyridyl)-1-(4-fluorophenyl)-1,2-dihydropyridin-2-one; 3-(2-cyanophenyl)-5-(2-pyridyl)-1-(4-formylphenyl)-1,2-dihydropyridin-2-one; 3-(2-cyanophenyl)-5-(2-pyridyl)-1-(2-formylphenyl)-1,2-dihydropyridin-2-one; 3-(2-cyanophenyl)-5-(2-pyridyl)-1-(3-chlorophenyl)-1,2-dihydropyridin-2-one; 3-(2-cyanophenyl)-5-(2-pyridyl)-1-(3-tolyl)-1,2-dihydropyridin-2-one; 3-(2-cyanophenyl)-5-(2-pyridyl)-1-(3-trifluoromethylphenyl)-1,2-dihydropyridin-2-one; 3-(2-cyanophenyl)-5-(2-pyridyl)-1-(thiophen-3-yl)-1,2-dihydropyridin-2-one; 3-(2-cyanophenyl)-5-(2-pyridyl)-1-(3-furfuryl)-1,2-dihydropyridin-2-one; 3-(2-cyanophenyl)-5-(2-pyridyl)-1-(4-tolyl)-1,2-dihydropyridin-2-one; 3-(2-cyanophenyl)-5-(2-pyridyl)-1-(4-trifluoromethylphenyl)-1,2-dihydropyridin-2-one; 3-(2-cyanophenyl)-5-(2-pyridyl)-1-(2-methoxypyridin-5-yl)-1,2-dihydropyridin-2-one; 3-(2-cyanophenyl)-5-(2-pyridyl)-1-(pyrimidin-5-yl)-1,2-dihydrop-yridin-2-one; 3-(2-cyanophenyl)-5-(2-pyridyl)-1-(3-benzyloxymethylpyridin-5-yl)-1,2-dihydropyridin-2-one; 3-(2-cyanophenyl)-5-(2-pyridyl)-1-(2-ethylthiopyridin-5-yl)-1,2-dihydropyridin-2-one; 3-(2-cyanophenyl)-5-(2-pyridyl-)-1-(4-pyridyl)-1,2-dihydropyridin-2-one; 3-(2-cyanophenyl)-5-(2-pyridyl)-1-(3-methoxypyridin-5-yl)-1,2-dihydropyridin-2-one; 3-(2-cyanophenyl)-5-(2-pyridyl)-1-(2-chloropyridin-5-yl)-1,2-dihydropyridin-2-one; 3-(2-cyanophenyl)-5-(2-pyridyl)-1-(2-fluoropyridin-5-yl)-1,2-dihydropyridin-2-one; 3-(2-cyanophenyl)-5-(2-pyridyl)-1-(2-methoxyphenyl)-1,2-dihydropyridin-2-one; 3-phenyl-5-(2-pyridyl)-1-(3-pyridyl)-1,2-dihydropyridin-2-one; 3-(2-chlorophenyl)-5-(2-pyridyl)-1-(3-pyridyl)-1,2-dihydropyridin-2-one; 3-(thiophen-3-yl)-5-(2-pyridyl)-1-(3-pyridyl)-1,2-dihydropyridin-2-one; 3-(2,6-dimethylphenyl)-5-(2-pyridyl)-1-(3-pyridyl)-1,2-dihydropyridin-2-one; 3-(2-cyanothiophen-3-yl)-5-(2-pyridyl)-1-(3-pyridyl)-1,2-dihydropyridin-2-one; 3-(2-fluoro-3-pyridyl)-5-(2-pyridyl)-1-(3-pyridyl)-1,2-dihydropyridin-2-one; 3-(2-chlorophenyl)-5-(2-pyridyl)-1-(3-hydroxyphenyl)-1-,2-dihydropyridin-2-one; 3-(2-chlorophenyl)-5-(2-pyridyl)-1-(3-dimethylaminoethoxyphenyl)-1,2-dihydropyridin-2-one; 3-(2-chlorophenyl)-5-(2-pyridyl)-1-(3-dimethylaminopropoxyphenyl)-1,2-dihydropyridin-2-one; 3-(2-cyanophenyl)-5-(2-pyridyl)-1-(2-hydroxymethylphenyl)-1,2-dihydropyridin-2-one; 3-(2-cyanophenyl)-5-(2-pyridyl)-1-(4-cyanomethylphenyl)-1,2-dihydropyridin-2-one; 3-(2-cyanophenyl)-5-(2-pyridyl)-1-(2-cyanomethylphenyl)-1,2-dihydropyridin-2-one; 3-(2-cyanophenyl)-5-(6-diethylaminomethyl-2-pyridyl)-1-phenyl-1,2-dihydropyridin-2-one; 3-(2-cyanophenyl)-1-phenyl-5-(2-pyrimidinyl)-1,2-dihydropyridin-2-one; 3-(2-hydroxypyridin-6-yl)-1-phenyl-5-(2-pyridyl)-1,2-dihydropyridin-2-one; 1-(2-aminobenzothiazol-6-yl)-3-(2-cyanophenyl)-5-(2-pyridyl)-1,2-dihydropyridin-2-one; 3-(2-cyanophenyl)-5-(2-pyridyl)-1-(1-benzyl-1,2,3,6-tetrahydropyridin-5-yl)-1,2-dihydropyridin-2-one; 3-[2-(5-methyl-1,2,4-oxadiazol-3-yl)phenyl]-1-phenyl-5-(2-pyridyl)-1,2-dihydropyridin-2-one; 3-(2-cyanophenyl)-5-(6-methylpyridin-2-yl)-1-phenyl-1,2-dihydropyridin-2-one; 3-(2-cyanophenyl)-5-(5-methylpyridin-2-yl)-1-phenyl-1,2-dihydropyridin-2-one; 3-(2-cyanophenyl)-5-(3-hydroxypyridin-2-yl)-1-phenyl-1,2-dihydropyridin-2-one; 3-(2-cyanophenyl)-1-phenyl-5-(2-thiazolyl)-1,2-dihydropyridin-2-one; 3-(2-cyanophenyl)-5-(2-methoxypyridin-6-yl)-1-phenyl-1,2-dihydropyridin-2-one; 1-(4-aminophenyl)-3-(2-cyanophenyl)-5-(2-pyridyl)-1,2-dihydropyridin-2-one; 1-(3-aminophenyl)-3-(2-cyanophenyl)-5-(2-pyrimidinyl)-1,2-dihydropyridin-2-one; 3-(2-cyanophenyl)-5-(2-pyridyl)-1-(2-aminotoluen-4-yl)-1-,2-dihydropyridin-2-one; 3-(2-cyanophenyl)-1-[3-(dimethylaminoethoxy)phenyl]-5-(2-pyridyl)-1,2-dihydropyridin-2-one; 3-(2-cyanophenyl)-1-[3-(piperidinoethoxy)phenyl]-5-(2-pyridyl)-1,2-dihydropyridin-2-one; 3-(2-cyanophenyl)-1-[3-(pyrrolidino ethoxy)phenyl]-5-(2-pyridyl)-1,2-dihyd-ropyridin-2-one; 3-(2-cyanophenyl)-1-[3-(diisoproylaminoethoxy)phenyl]-5-(-2-pyridyl)-1,2-dihydropyridin-2-one; 3-(2-cyanophenyl)-1-[3-(4-piperidinobutyl-1-oxy)phenyl]-5-(2-pyridyl)-1,2-dihydropyridin-2-one; 3-(2=cyanophenyl)-1-(4-nitrophenyl)-5-(2-pyridyl)-1,2-dihydropyridin-2-one; 1-phenyl-5-(2-pyridyl)-3-(2-thiazolyl)-1,2-dihydropyridin-2-one; 3-(2-cyanophenyl)-1-(3-pyridyl)-5-(2-pyrimidinyl)-1,2-dihydropyridin-2-one; 3-(2-fluoropyridin-3-yl)-1-phenyl-5-(2-pyrimidinyl)-1,2-dihydropyridin-2-one; 3-(2-cyanopyridin-3-yl)-1-phenyl-5-(2-pyrimidinyl)-1,2-dihydropyridin-2-one; 3-(2-cyanophenyl)-1-(3-nitrophenyl)-5-(2-pyrimidinyl)-1,2-dihydropyridin-2-one; 3-(2-nitrophenyl)-1-phenyl-5-(2-pyridyl)-1,2-dihydropyridin-2-one; 3-(2-formylthiophen-3-yl)-5-(2-pyridyl)-1-phenyl-1,2-dihydropyrid-in-2-one; 3-(2-cyanophenyl)-5-(2-pyridyl)-1-(2-naphthyl)-1,2-dihydropyridin-2-one; 3-(2-cyanophenyl)-5-(2-pyridyl)-1-(1-naphthyl)-1,2-dihydropyridin-2-one; 5-(2-aminopyridin-6-yl)-3-(2-cyanophenyl)-1-phenyl-1,2-dihydropyridin-2-one; 5-(2-bromopyridin-6-yl)-3-(2-cyanophenyl)-1-phenyl-1,2-dihydropyridin-2-one; 3-(2-cyanophenyl)-5-(2-morphorinopyridin-6-yl)-1-phenyl-1,2-dihydropyridin-2-one; 3-(2-cyanophenyl)-1-(3-hydroxyphenyl)-5-(2-pyridyl)-1,2-dihydropyridin-2-one; 3-(2-cyanophenyl)-1-[3-(4-piperidyloxy)]phenyl-5-(2-pyridyl)-1,2-dihydropyridin-2-one; 1-[3-(N-acetylpiperidin-4-yl-oxy)phenyl]-3-(2-cyanophenyl)-5-(2-pyridyl)-1,2-dihydropyridin-2-one; 3-(2-cyanophenyl)-1-{3-[1-(methanesulfonyl)piperidin-4-yl-oxy]phenyl}-5-(2-pyridyl)-1,2-dihydropyridin-2-one; 1-[3-(N-methylpiperidin-4-yl-oxy)pheny-1]-3-(2-cyanophenyl)-5-(2-pyridyl)-1,2-dihydropyridin-2-one; 3-(6-chloro-1H-benzimidazol-2-yl)-5-(2-pyridyl)-1-phenyl-1,2-dihydropyridin-2-one; 3-(2-cyanophenyl)-5-(2-pyridyl)-1-(2-nitrotoluen-4-yl)-1,2-dihydropyridin-2-one; 3-(2-cyanothiophen-3-yl)-5-(2-pyridyl)-1-phenyl-1,2-dihydropyridin-2-one; 3-[2-(5-oxazolyl)phenyl]-1-phenyl-5-(2-pyridyl)-1,2-dihydropyridin-2-one; 3-[2-(5-oxazoly)thiophen-3-yl]-1-phenyl-5-(2-pyridyl)-1,2-dihydropyridin-2-one; and 3-(2-ethoxycarbonylvinylthiophen-3-yl)-5-(2-pyridyl)-1-phenyl-1,2-dihydropyridin-2-one.

The 1,2-dihydropyridine compounds and methods for making the 1,2-dihydropyridine compounds are described in U.S. Pat. No. 6,949,571, US Publication No. 2004/0023973, and PCT Publication Nos. WO 03/047577, WO 04/009553, WO 06/004100, WO 06/004107, WO07/072,868, and WO07/072,869, the disclosures of which are incorporated by reference herein in their entirety.

Methods for administering, dosing, and making other AMPA receptor antagonists such as quinoxalinedione aminoalkylphosphonates are described, for example, in WO 2005/094797 and WO 98/17672.

In one embodiment, the compounds used in the methods and compositions described herein are cholinesterase inhibitors. The cholinesterase inhibitors may be any known in the art. The term “cholinesterase inhibitor” includes cholinesterase inhibitors, pharmaceutically acceptable salts of cholinesterase inhibitors, stereoisomers of cholinesterase inhibitors, and pharmaceutically acceptable salts of stereoisomers of cholinesterase inhibitors. Exemplary cholinesterase inhibitors include donepezil, tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, epastigmine, upreazine, 3-[1-(phenylmethyl)-4-piperidinyl]-1-(2,3,4,5-tetrahydro-1H-1-benzazepin-8-yl)-1-propanone, (2-[2-(1-benzylpiperidin-4-yl)ethyl]-2,3-dihydro-9-methoxy-1H-pyrrolo[3,4-b]quinolin-1-one), and the like. In one embodiment, the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof; and galanthamine or a pharmaceutically acceptable salt thereof. In one embodiment, the cholinesterase inhibitor is donepezil, tacrine, galantamine, or rivastigmine.

In one embodiment, the cholinesterase inhibitors used in the methods and compositions described herein are compounds of formula (IV):

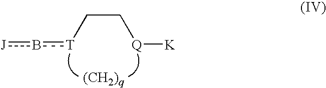

wherein J is (a) a substituted or unsubstituted group selected from (i) phenyl, (ii) pyridyl, (iii) pyrazyl, (iv) quinolyl, (v) cyclohexyl, (vi) quinoxalyl, and (vii) furyl; (b) a monovalent or divalent group, in which the phenyl can have one or more substituents selected from (i) indanyl, (ii) indanonyl, (iii) indenyl, (iv) indenonyl, (v) indanedionyl, (vi) tetralonyl, (vii) benzosuberonyl, (viii) indanolyl, and (ix) C₆H₅—CO—CH(CH₃)—; (c) a monovalent group derived from a cyclic amide compound; (d) a lower alkyl; or (e) R²¹—CH═CH—, in which R²¹ is hydrogen or a lower alkoxycarbonyl; B is —(CHR²²)ᵣ—, —CO—(CHR²²)ᵣ—, —NR⁴—(CHR²²)ᵣ—, —CO—NR⁵—(CHR²²)ᵣ—, —CH═CH—(CHR²²)ᵣ—, —OCOO—(CHR²²)ᵣ—, —OOC—NH—(CHR²²)ᵣ—, —NH₂—CO—NH—(CHR²²)ᵣ—, —(CH₂)ᵣ—NH—(CHR²²)ᵣ—, —CH(OH)—(CHR²²)ᵣ—, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbonyl; R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of 1 to 10; R²² is hydrogen or methyl so that one alkylene group can have no methyl branch or one or more methyl branches; b is an integer of 1 to 3; c is zero or an integer of 1 to 9; d is zero or an integer of 1 to 5; T is nitrogen or carbon; Q is nitrogen, carbon or

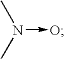

q is an integer of 1 to 3; K is hydrogen, phenyl, substituted phenyl, arylalkyl in which the phenyl can have a substituent, cinnamyl, a lower alkyl, pyridylmethyl, cycloalkylalkyl, adamantanemethyl, furylmethyl, cycloalkyl, lower alkoxycarbonyl or an acyl; and [symbol] is a single bond or a double bond.

In the compound of formula (IV), J is preferably (a) or (b), more preferably (b). In the definition of (b), a monovalent group (2), (3) and (5) and a divalent group (2) are preferred. The group (b) preferably includes the formulae shown below:

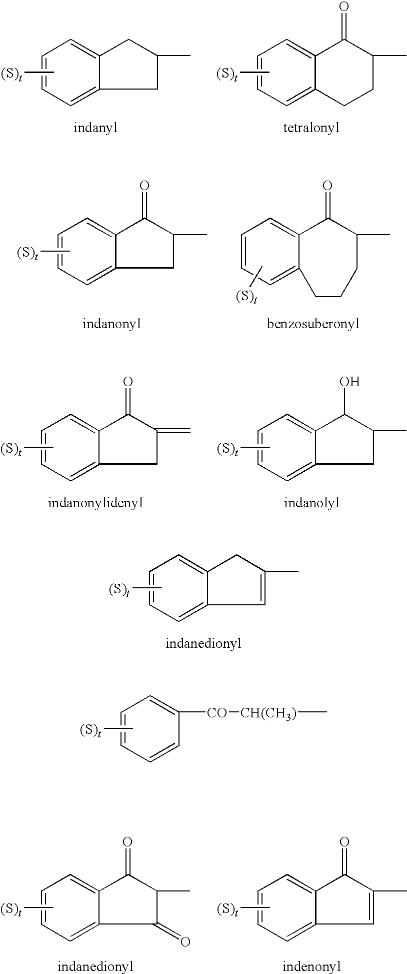

wherein t is an integer of 1 to 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy thereon. (S)ₜ can form methylene dioxy or ethylene dioxy on two adjacent carbon atoms of the phenyl. Of the above groups, indanonyl, indanedionyl and indenyl, optionally having substituents on the phenyl, are the most preferred.

In the definition of B, —(CHR²²)ᵣ—, —CO—(CHR²²)ᵣ—, ═(CH—CH═CH)_{b}—, ═CH—(CH₂)_{c}— and ═(CH—CH)_{d}═ are preferable. The group —(CHR²²)ᵣ— in which R²² is hydrogen and r is an integer of 1 to 3, and the group ═CH—(CH₂)_{c}— are most preferable. The preferable groups of B can be connected with (b) of J, in particular (b)(2). The ring containing T and Q in formula (I) can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2. It is preferable that K is a phenyl, arylalkyl, cinnamyl, phenylalkyl or a phenylalkyl having a substituent(s) on the phenyl.

In one embodiment, the cholinesterase inhibitors used in the methods and compositions described herein are compounds of formula (V):

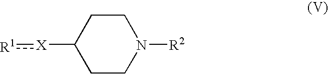

wherein R¹ is a (1) substituted or unsubstituted phenyl; (2) a substituted or unsubstituted pyridyl; (3) a substituted or unsubstituted pyrazyl; (4) a substituted or unsubstituted quinolyl; (5) a substituted or unsubstituted indanyl; (6) a substituted or unsubstituted cyclohexyl; (7) a substituted or unsubstituted quinoxalyl; (8) a substituted or unsubstituted furyl; (9) a monovalent or divalent group derived from an indanone having a substituted or unsubstituted phenyl; (10) a monovalent group derived from a cyclic amide compound; (11) a lower alkyl; or (12) R³—CH═C—, where R³ is a hydrogen atom or a lower alkoxycarbonyl; X is —(CH₂)ₙ—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl, a lower alkoxycarbonyl; n is an integer of 0 to 6; R⁴ is hydrogen, lower alkyl, acyl, lower alkylsulfonyl, a substituted or unsubstituted phenyl, or a substituted or unsubstituted benzyl; and R⁵ is hydrogen, lower alkyl, or phenyl; R² is a substituted or unsubstituted phenyl; a substituted or unsubstituted arylalkyl; a cinnamyl; lower alkyl; pyridylmethyl; cycloalkylalkyl; adamantanemethyl; or furoylmethyl; and [symbol] is a single bond or a double bond.

With respect to the cholinesterase inhibitors described herein, the term “lower alkyl” means a straight or branched alkyl having 1 to 6 carbon atoms. Exemplary “lower alkyl” groups include methyl, ethyl, propyl, isopropyl, butyl, isobutyl, sec-butyl, tert-butyl, pentyl (amyl), isopentyl, neopentyl, tent-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimthyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl is preferably methyl, ethyl, propyl or isopropyl; more preferably methyl.

With respect to the cholinesterase inhibitors described herein, specific examples of the substituents for the substituted or unsubstituted phenyl, pyridyl, pyrazyl, quinolyl, indanyl, cyclohexyl, quinoxalyl and furyl in the definition of R¹ include lower alkyl groups having 1 to 6 carbon atoms, such as methyl, ethyl, n-propyl, isopropyl, n-butyl, isobutyl, and tert-butyl groups; lower alkoxy corresponding to the above-described lower alkyl, such as methoxy and ethoxy groups; nitro; halogen, such as chlorine, fluorine and bromine; carboxyl; lower alkoxycarbonyl corresponding to the above-described lower alkoxy, such as methoxycarbonyl, ethoxycarbonyl, isopropoxycarbonyl, n-propoxycarbonyl, and n-butyloxycarbonyl groups; amino; lower monoalkylamino; lower dialkylamino; carbamoyl; acylamino derived from aliphatic saturated monocarboxylic acids having 1 to 6 carbon atoms, such as acetylamino, propionylamino, butyrylamino, isobutyrylamino, valerylamino, and pivaloylamino; cycloalkyloxycarbonyl, such as a cyclohexyloxycarbonyl; lower alkylaminocarbonyl, such as methylaminocarbonyl and ethylaminocarbonyl; lower alkylcarbonyloxy corresponding to the above-defined lower alkyl, such as methylcarbonyloxy, ethylcarbonyloxy, and n-propylcarbonyloxy; halogenated lower alkyl, such as trifluoromethyl; hydroxyl; formyl; and lower alkoxy lower alkyl, such as ethoxymethyl, methoxymethyl and methoxyethyl. The “lower alkyl” and “lower alkoxyl” in the above description of the substituents include all the groups derived from the above-mentioned groups. There may be 1, 2 or 3 substituents, which may be the same or different.

With respect to the cholinesterase inhibitors described herein, when the substituent is phenyl, the following is within the scope of the substituted phenyl:

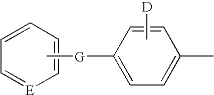

wherein G is —C(O)—, —O—C(O)—, —O—, —CH₂—NH—C(O)—, —CH₂—O—, —CH₂—SO₂—, —CH(OH)—, or —CH₂—S(→O)—; E is carbon or nitrogen; and D is a substituent.

With respect to the cholinesterase inhibitors described herein, preferred examples of the substituents (i.e., “D”) for the phenyl include lower alkyl, lower alkoxy, nitro, halogenated lower alkyl, lower alkoxycarbonyl, formyl, hydroxyl, and lower alkoxy lower alkyl, halogen, and benzyol and benzylsulfonyl. There may be 2 or more substituents, which may be the same or different. Preferred examples of the substituent for the pyridyl include lower alkyl and amino and halogen. Preferred examples of the substituent for the pyrazyl include lower alkoxycarbonyl, carboxyl, acylamino, carbamoyl, and cycloalkyloxycarbonyl.

With respect to the cholinesterase inhibitors described herein, with respect to R¹, the pyridyl is preferably a 2-pyridyl, 3-pyridyl, or 4-pyridyl; the pyrazyl is preferably a 2-pyrazinyl; the quinolyl is preferably a 2-quinolyl or 3-quinolyl; the quinoxalinyl is preferably a 2-quinoxalinyl or 3-quinoxalinyl; and the furyl is preferably a 2-furyl.

With respect to the cholinesterase inhibitors described herein, examples of monovalent or divalent groups derived from an indanone having an unsubstituted or substituted phenyl of (A) or (B):

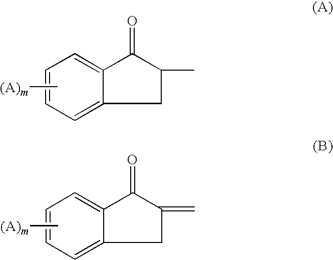

where m is an integer of from 1 to 4, and each A is independently hydrogen, lower alkyl, lower alkoxy, nitro, halogen, carboxyl, lower alkoxycarbonyl, amino, lower monoalkylamino, lower dialkylamino, carbamoyl, acylamino derived from aliphatic saturated monocarboxylic acids having 1 to 6 carbon atoms, cycloalkyloxycarbonyl, lower alkylaminocarbonyl, lower alkylcarbonyloxy, halogenated lower alkyl, hydroxyl, formyl, or lower alkoxy lower alkyl; preferably hydrogen, lower alkyl, or lower alkoxy; most preferably the indanone is unsubstituted or substituted with 1 to 3 methoxy.

With respect to the cholinesterase inhibitors described herein, examples of the monovalent group derived from a cyclic amide compound include quinazolone, tetrahydroisoquinolinone, tetrahydrobenzodiazepinone, and hexahydrobenzazocinone. The monovalent group can be any one having a cyclic amide in the structural formula thereof, and is not limited to the above-described specific examples. The cyclic amide can be one derived from a monocyclic or condensed heterocyclic ring. The condensed heterocyclic ring is preferably one formed by condensation with phenyl. In this case, phenyl can be substituted with a lower alkyl group having 1 to 6 carbon atoms, preferably methyl, or lower alkoxy having 1 to 6 carbon atoms, preferably methoxy.

With respect to the cholinesterase inhibitors described herein, examples of the monovalent group include the following:

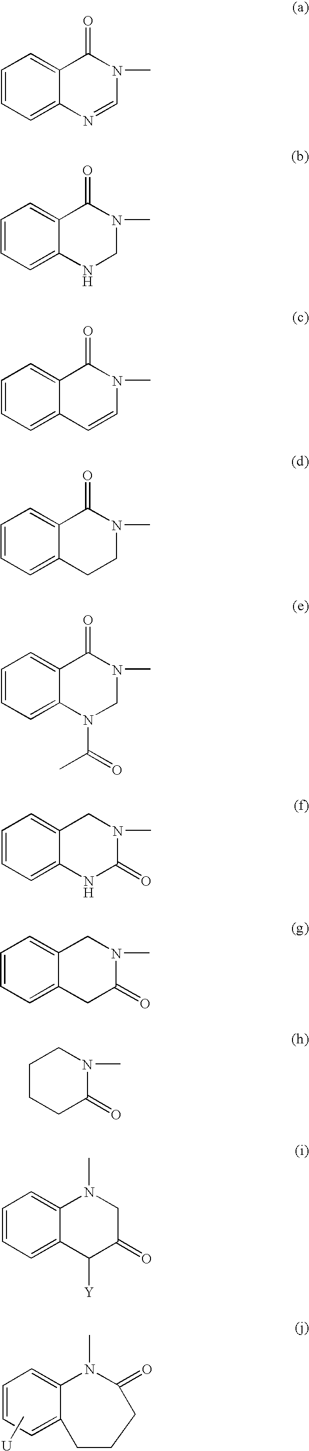

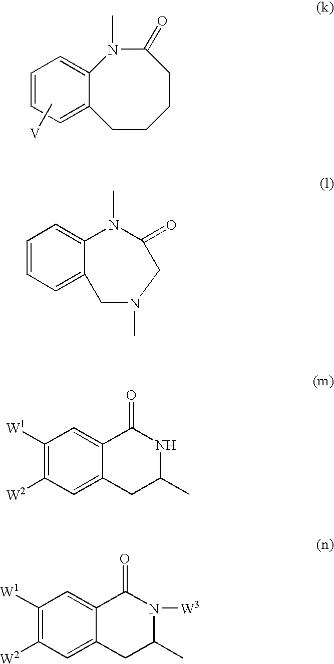

In the above formulae, Y is hydrogen or lower alkyl; V and U are each hydrogen or lower alkoxy (preferably dimethoxy); W¹ and W² are each hydrogen, lower alkyl, or lower alkoxy; and W³ is hydrogen or a lower alkyl. The right hand ring in formulae (j) and (l) is a 7-membered ring, while the right hand ring in formula (k) is an 8-membered ring.

With respect to the cholinesterase inhibitors described herein, the most preferred examples of R¹ include a monovalent group derived from an indanone having an unsubstituted or substituted phenyl and a monovalent group derived from a cyclic amide compound. With respect to the cholinesterase inhibitors described herein, the most preferred examples of X include —(CH₂)ₙ—, amide, or groups represented by the above formulae where n is 2. Thus, it is most preferred that any portion of a group represented by

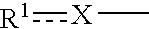

have a carbonyl or amide.

With respect to the cholinesterase inhibitors described herein, the substituents involved in the expressions “a substituted or unsubstituted phenyl” and “a substituted or unsubstituted arylalkyl” in the above definition of R² are the same substituents as those described for the above definitions of phenyl, pyridyl, pyrazyl, quinolyl, indanyl, cyclohexyl, quinoxalyl or furyl in the definition of R¹. The term “arylalkyl” is intended to mean an unsubstituted benzyl or phenethyl or the like. Specific examples of the pyridylmethyl include 2-pyridylmethyl, 3-pyridylmethyl, and 4-pyridylmethyl. Preferred examples of R² include benzyl and phenethyl. The symbol [symbol] means a double or single bond. The bond is a double bond only when R¹ is the divalent group (B) derived from an indanone having an unsubstituted or substituted phenyl, while it is single bond in other cases.

In one embodiment, the cholinesterase inhibitors used in the methods and compositions described herein are compounds of formula (VI):

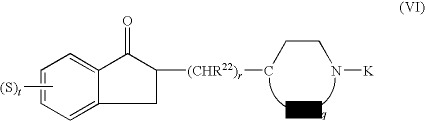

wherein r is an integer of 1 to 10; each R²² is independently hydrogen or methyl; K is a phenalkyl or a phenalkyl having a substituent on the phenyl; each S is independently hydrogen, C₁₋₆ lower alkyl, or C₁₋₆ lower alkoxy; t is an integer of 1 to 4; q is an integer of 1 to 3; with the proviso that (S)ₜ can be methylenedioxy or ethylenedioxy joined to two adjacent carbon atoms of the phenyl.

In other embodiments, the compound of formula (VI) is 1-benzyl-4-((5,6-dimethoxy-1-indanon)-2-yl)methylpiperidine; 1-benzyl-4-((5,6-dimethoxy-1-indanon)-2-ylindenyl)methylpiperidine; 1-benzyl-4-((5-methoxy-1-indanon)-2-yl)methylpiperidine; 1-benzyl-44(5,6-diethoxy-1-indanon)-2-yl)methylpiperidine; 1-benzyl-44(5,6-methylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy-1-indanon)-2-yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; pharmaceutically acceptable salts of one or more of the foregoing; stereoisomers of one or more of the foregoing; or pharmaceutically acceptable salts of stereoisomers of one or more of the foregoing.

In other embodiments, the compound of formula (VI) used in the methods and compositions described herein is 1-benzyl-4-((5,6-dimethoxy-1-indanon)-2-yl)methylpiperidine; which is represented by formula (B):

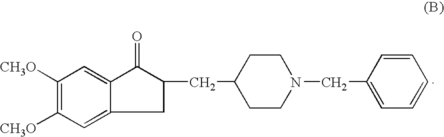

The compound of Formula (B), known as donepezil, may be in the form of a polymorph or a polymorphic crystal. For example, donepezil may be in the form of polymorph (II), (III), (IV), or (V); preferably polymorph (H). Donepezil may be in the form of polymorphic crystals (A), (B), or (C). Polymorphs, polymorphic crystals, and methods for making polymorphs and polymorphic crystals are described in U.S. Pat. Nos. 5,985,864, 6,140,321 and 6,245,911, the disclosures of which are incorporated by reference herein in their entirety.

In still other embodiments, the compound of formula (III) is 1-benzyl-4-((5,6-dimethoxy-1-indanon)-2-yl)methylpiperidine hydrochloride, which is also known as donepezil hydrochloride, and which is represented by formula (B1):

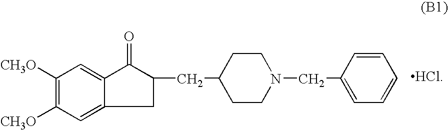

The compounds of the invention can have an asymmetric carbon atom(s), depending upon the substituents, and can have stereoisomers, which are within the scope of the invention. For example, donepezil or pharmaceutically acceptable salts thereof can be in the forms described in Japanese Patent Application Nos. 4-187674 and 4-21670, the disclosures of which are incorporated by reference herein in their entirety.

Japanese Patent Application No. 4-187674 describes a compound of formula (B2):

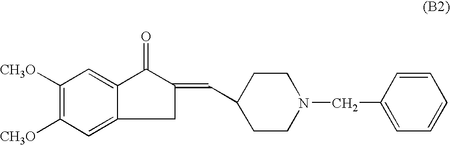

which can be in the form of a pharmaceutically acceptable salt, such as a hydrochloride salt.

Japanese Patent Application No. 4-21670 describes compounds of formula (B3):

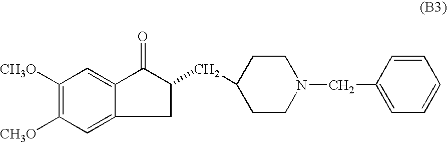

which can be in the form of a pharmaceutically acceptable salt, such as a hydrochloride salt; and compounds of formula (B4):

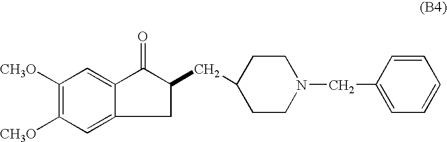

which can be in the form of a pharmaceutically acceptable salt, such as a hydrochloride salt; and compounds of formula (B5):

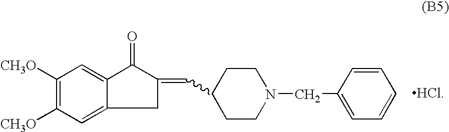

Throughout the specification, the terms “donepezil” and “1-benzyl-4-((5,6-dimethoxy-1-indanon)-2-yl)methylpiperidine” are intended to include one or more of the following (e.g., combinations of two or more thereof): pharmaceutically acceptable salts; stereoisomers; polymorphs; and polymorphic crystals.

The cholinesterase inhibitors of the invention are commercially available or can be prepared by processes known in the art, such as those described, for example, in U.S. Pat. Nos. 4,895,841, 5,985,864, 6,140,321 and 6,245,911; WO 98/39000, and Japanese Patent Application Nos. 4-187674 and 4-21670, the disclosures of which are incorporated by reference herein in their entirety.

The anti-neuropathic pain agents used in the methods and compositions of the invention may be any known in the art. Anti-neuropathic pain agents include, for example, sodium channel blockers, calcium channel inhibitors/modulators, antidepressant, anticonvulsant, antiarrhythmics, glutamate antagonists, muscle relaxant, antiepileptics, NMDA antagonists, cannabinoid receptor agonists, opioids, acetylcholine receptor agonists, alpha2 adrenaline agonists, CGRP antagonists and TRPV1 ligands. Other exemplary anti-neuropathic pain agents include gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818. In one embodiment, the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818. Anti-neuropathic pain agents are commercially available or can be prepared by methods well known in the literature.

In other embodiments, the invention provides pharmaceutical compositions comprising a therapeutically effective amount of: (i) at least one AMPA receptor antagonist, (ii) at least one cholinesterase inhibitor, and (iii) at least one pharmaceutically acceptable excipient. The invention also provides combinations comprising a therapeutically effective amount of: (i) at least one AMPA receptor antagonist and (ii) at least one cholinesterase inhibitor; wherein the compounds may be administered separately (e.g., simultaneously, sequentially) to a patient to treat the diseases or disorders described herein. The invention provides kits (e.g., commercial packages) comprising a therapeutically effective amount of: (i) at least one AMPA receptor antagonist, (ii) at least one cholinesterase inhibitor; and (iii) instructions for the simultaneous, separate or sequential use of (i) and (ii) in the treatment of the diseases and disorders described herein. The AMPA receptor antagonist can be any described herein. For example, the 1,2-dihydropyridine compound can be a compound of Formula (I), a compound of Formula (II), a compound of Formula (III), or Compound A. The cholinesterase inhibitor can be any described herein. For example, the cholinesterase inhibitor can be a compound of Formula (IV), a compound of Formula (V), a compound of Formula (VI), a compound of Formula (B), a compound of Formula (B1), a compound of Formula (B2), a compound of Formula (B3), a compound of Formula (B4), or a compound of Formula (B5). In other embodiments, the cholinesterase inhibitor can be tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, epastigmine, upreazine, 3-[1-(phenylmethyl)-4-piperidinyl]-1-(2,3,4,5-tetrahydro-1H-1-benzazepin-8-yl)-1-propanone, or (2-[2-(1-benzylpiperidin-4-yl)ethyl]-2,3-dihydro-9-methoxy-1H-pyrrolo[3,4-b]quinolin-1-one. In one embodiment, the invention provides pharmaceutical compositions comprising a therapeutically effective amount of: (i) donepezil; (ii) 3-(2-cyanophenyl)-5-(2-pyridyl)-1-phenyl-1,2-dihydropyridin-2-one; and (iii) at least one pharmaceutically acceptable excipient. The pharmaceutical compositions, combination and kits can optionally further comprise at least one anti-neuropathic pain agent.

The invention provides pharmaceutical compositions comprising a therapeutically effective amount of: (i) at least one AMPA receptor antagonist, (ii) at least one anti-neuropathic pain agent, and (iii) at least one pharmaceutically acceptable excipient. The invention also provides combinations comprising a therapeutically effective amount of: (i) at least one AMPA receptor antagonist and (ii) at least one anti-neuropathic pain agent; wherein the compounds may be administered separately (e.g., simultaneously, sequentially) to a patient to treat the diseases or disorders described herein. The invention provides kits (e.g., commercial packages) comprising a therapeutically effective amount of: (i) at least one AMPA receptor antagonist, (ii) at least one anti-neuropathic pain agent; and (iii) instructions for the simultaneous, separate or sequential use of (i) and (ii) in the treatment of the diseases and disorders described herein. The AMPA receptor antagonist can be any described herein. For example, the 1,2-dihydropyridine compound can be a compound of Formula (I), a compound of Formula (II), a compound of Formula (III), or a compound of Formula (A). The anti-neuropathic pain agent can be any described herein. For example, the anti-neuropathic pain agent can be gabapentin, pregabalin, duloxetine, amitriptyline, or dextromethorphan. The pharmaceutical compositions, combination and kits can optionally further comprise at least one cholinesterase inhibitor.

In other embodiments, the invention provides pharmaceutical compositions comprising a therapeutically effective amount of: (i) at least one AMPA receptor antagonist, (ii) at least one cholinesterase inhibitor, (iii) at least one anti-neuropathic pain agent, and (iv) at least one pharmaceutically acceptable excipient. The invention also provides combinations comprising a therapeutically effective amount of: (i) at least one AMPA receptor antagonist, (ii) at least one cholinesterase inhibitor and (iii) at least one anti-neuropathic pain agent; wherein the compounds may be administered separately (e.g., simultaneously, sequentially) to a patient to treat the diseases or disorders described herein. The invention provides kits (e.g., commercial packages) comprising a therapeutically effective amount of: (i) at least one AMPA receptor antagonist, (ii) at least one cholinesterase inhibitor, (iii) at least one anti-neuropathic pain agent; and (iv) instructions for the simultaneous, separate or sequential use of (i) to (iii) in the treatment of the diseases and disorders described herein. The AMPA receptor antagonist can be any described herein. For example, the 1,2-dihydropyridine compound can be a compound of Formula (I), a compound of Formula (II), a compound of Formula (III), or Compound A. The cholinesterase inhibitor can be any described herein. For example, the cholinesterase inhibitor can be a compound of Formula (IV), a compound of Formula (V), a compound of Formula (VI), a compound of Formula (B), a compound of Formula (B1), a compound of Formula (B2), a compound of Formula (B3), a compound of Formula (B4), or a compound of Formula (B5). In other embodiments, the cholinesterase inhibitor can be tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, epastigmine, upreazine, 3-[1-(phenylmethyl)-4-piperidinyl]-1-(2,3,4,5-tetrahydro-1H-1-benzazepin-8-yl)-1-propanone, or (2-[2-(1-benzylpiperidin-4-yl)ethyl]-2,3-dihydro-9-methoxy-1H-pyrrolo[3,4-b]quinolin-1-one. The anti-neuropathic pain agent can be any described herein. For example, the anti-neuropathic pain agent can be gabapentin, pregabalin, duloxetine, amitriptyline, or dextromethorphan. In one embodiment, the invention provides pharmaceutical compositions comprising a therapeutically effective amount of (i) donepezil; (ii) 3-(2-cyanophenyl)-5-(2-pyridyl)-1-phenyl-1,2-dihydropyridin-2-one, (iii) at least one anti-neuropathic pain agent; and (iv) at least one pharmaceutically acceptable excipient.

The invention provides methods for the treatment and/or prophylaxis of neuropathic pain in a patient in need thereof by administering a therapeutically effective amount of: (a) at least one AMPA receptor antagonist, and (b); wherein (b) is a therapeutically effective amount of (i) at least one cholinesterase inhibitor; (ii) at least one anti-neuropathic pain agent; or (iii) at least one cholinesterase inhibitor and at least one anti-neuropathic pain agent. The methods for the treatment of neuropathic pain include (i) methods for reducing the frequency of neuropathic pain, (ii) methods for reducing the severity of neuropathic pain, (iii) methods for reducing the duration of neuropathic pain, (iv) methods for reducing the frequency and severity of neuropathic pain, (v) methods for reducing the frequency and duration of neuropathic pain, (vi) methods for reducing the severity and duration of neuropathic pain, and (vii) methods for reducing the frequency, severity and duration of neuropathic pain. The methods for the treatment of neuropathic pain includes methods of treating one or more symptoms caused by neuropathic pain. The AMPA receptor antagonist and the cholinesterase inhibitor and/or anti-neuropathic pain agent can be administered separately to the patient or may be administered in the form of a pharmaceutical composition.

The invention provides methods for the treatment and/or prophylaxis of diabetic neuropathy in a patient by administering a therapeutically effective amount of: (a) at least one AMPA receptor antagonist, and (b); wherein (b) is a therapeutically effective amount of: (i) at least one cholinesterase inhibitor; (ii) at least one anti-neuropathic pain agent; or (iii) at least one cholinesterase inhibitor and at least one anti-neuropathic pain agent. The methods for treating diabetic neuropathy include (i) methods for reducing the frequency of diabetic neuropathy, (ii) methods for reducing the severity of diabetic neuropathy, (iii) methods for reducing the duration of diabetic neuropathy, (iv) methods for reducing the frequency and severity of diabetic neuropathy, (v) methods for reducing the frequency and duration of diabetic neuropathy, (vi) methods for reducing the severity and duration of diabetic neuropathy, and (vii) methods for reducing the frequency, severity and duration of diabetic neuropathy. The methods for the treatment of diabetic neuropathy include methods of treating of one or more symptoms caused by diabetic neuropathy. Diabetic neuropathy may also be referred to as painful diabetic neuropathy or diabetic neuropathic pain. Diabetic neuropathy is common complication of diabetes. It causes autonomic and sensory problems to the patients. Diabetic neuropathic pain is found in about 10% of the diabetic population. Diabetic neuropathic pain can be spontaneous or stimulus induced, severe or intractable. Diabetic neuropathic pain can be described as burning, pins and needles, shooting, aching, jabbing, sharpe, cramping, tingling, cold or allodynia.

The dosage form of the formulation included in the combination, kit and/or pharmaceutical composition of the invention is not particularly limited. The combination, kit and/or pharmaceutical composition of the invention is useful as a combination, kit and/or a pharmaceutical composition for treating neuropathic pain; for the prophylaxis of neuropathic pain; and for delaying the onset of neuropathic pain.

The combination, kit and/or pharmaceutical composition of the invention may be used as a drug for treating neuropathic pain; for the prophylaxis of neuropathic pain; and for delaying the onset of neuropathic pain.

The combination, kit and/or pharmaceutical composition of the invention may be administered to a patient.

The combination, kit and/or pharmaceutical composition of the invention may be used through oral or parenteral administration. When the combination, kit and/or pharmaceutical composition of the invention is used, the given dose of the compound of the invention differs depending on the degree of the symptom, age, sex, weight and sensitivity difference of the patient, administration mode, administration period, administration interval, nature, prescription and the type of the pharmaceutical formulation, and the type of the active element.

The pharmaceutical composition of the invention may be made into various forms, for example, into solid oral formulations, injectable solution or the like.

The AMPA receptor antagonists and other compounds of the invention (e.g., cholinesterase inhibitors, anti-neuropathic pain agents) can be administered orally, topically, parenterally, by inhalation (nasal or oral), or rectally in dosage unit formulations containing conventional nontoxic pharmaceutically acceptable carriers, adjuvants, and vehicles as desired. The term parenteral includes subcutaneous, intravenous, intramuscular, intrathecal, intrasternal injection, or infusion techniques.

The daily dose of the AMPA receptor antagonists of the invention (e.g., 3-(2-cyanophenyl)-5-(2-pyridyl)-1-phenyl-1,2-dihydropyridin-2-one) is usually 30 μg/day to 10 g/day; 100 μg/day to 5 g/day; or 100 μg/day to 100 mg/day in the case of oral administration. For administration by injection, the daily dose is usually 30 μg/day to 1 g/day; 100 μg/day to 500 mg/day; or 100 μg/day to 30 mg/day. The compounds are administered once daily or in several portions a day. When used in the context of a dosage amount, the numerical weight refers to the weight of the 1,2-dihydropyridine, exclusive of any salt, counterion, hydrate, and the like. Therefore to obtain the equivalent of 500 mg of 3-(2-cyanophenyl)-5-(2-pyridyl)-1-phenyl-1,2-dihydropyridin-2-one, it would be necessary to use more than 500 mg of a pharmaceutically acceptable salt and/or hydrate of the compound, due to the additional weight of the pharmaceutically acceptable salt and/or hydrate.

The daily dose of the cholinesterase inhibitors of the invention (e.g., donepezil) are usually 0.1 mg/day to 100 mg/day; 1 mg/day to 50 mg/day; or 3 mg/day to 25 mg/day. In other embodiment, the daily dose is from 10 mg/day to 20 mg/day; or from 3 mg/day to 10 mg/day. The compounds are administered once daily or in several portions a day. When used in the context of a dosage amount, the numerical weight refers to the weight of the cholinesterase inhibitors of the invention, exclusive of any salt, counterion, and the like. Therefore to obtain the equivalent of 10 mg of donepezil, it would be necessary to use more than 10 mg of donepezil hydrochloride, due to the additional weight of the hydrochloride.

The daily dose of the anti-neuropathic pain agent of the invention (e.g., antidepressant including duloxetine, amitriptyline or the others, anticonvulsant including gabapentin, pregabalin, carbamazepine or the others, antiarrhythmics including lidocaine, mexiletine or the others, glutamate antagonists including ketamine, memantine and the others, muscle relaxant including baclofen, tizanidine or the others or opioids including morphine, fentanyl or the others) are usually 1 mg/day to 300 mg/day for antidpressant; 0.5 mg/day to 4000 mg/day for anticonvulsant; or 50 mg/day to 1000 mg/day for antiarrhythmics, 5 mg/day to 200 mg/day for glutamate antagonists, 1 mg/day to 200 mg/day for muscle relaxants or 0.5 mg/day to 300 mg/day for opioids. The compounds are administered once daily, in several portions a day or continuously. When used in the context of a dosage amount, the numerical weight refers to the weight of the anti-neuropathic pain agents of the invention, exclusive of any salt, counterion, and the like. Therefore to obtain the equivalent of anti-neuropathic pain drugs, it would be necessary to use more than above noted amount of drugs, due to the additional weight of the salts.

When administered to a child, the dose may possibly be lower than that for an adult. The actual method for administration may fluctuate widely and may depart from the preferred method described herein.

The other compounds described herein (e.g., anti-neuropathic pain agents) may be administered in doses well known in the art by reference, for example, to The Physician's Desk Reference, to patents describing doses for the compounds, and to journal articles describing doses for the compounds.

In one embodiment, the mode of administration is by injection, such as subcutaneous injection, intramuscular injection, intravenous injection, or intra-arterial injection. Injectable preparations, for example, sterile injectable aqueous or oleaginous suspensions can be formulated according to the art using suitable dispersing or wetting agents, suspending agents (e.g., methylcellulose, Polysorbate 80, hydroxyethylcellulose, acacia, powdered tragacanth, sodium carboxymethylcellulose, polyoxyethylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), stabilizers (e.g., sodium sulfite and sodium metasulfite; examples of the preservative include methyl parahydroxybenzoate, ethyl parahydroxybenzoate, sorbic acid, phenol, cresol, chlorocresol, and the like), tonicity agents and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

In order to prepare a solid oral formulation, an excipient, and if necessary, a binder, disintegrant, lubricant, colorant, a flavoring agent and the like are added to the principal agent, and then made into a tablet, a coated tablet, granule, fine granule, dispersant, a capsule or the like according to a conventional method.

For example, lactose, cornstarch, sucrose, glucose, sorbit, crystalline cellulose, silicon dioxide or the like may be used as the excipient; for example, polyvinyl alcohol, ethyl cellulose, methyl cellulose, gum arabic, hydroxypropyl cellulose, hydroxypropylmethyl cellulose or the like may be used as the binder; for example, magnesium stearate, talc, silica or the like may be used as the lubricant; those that are allowed to be added to drugs may be used as the colorant; and for example, cocoa powder, menthol, aromatic acid, peppermint oil, camphor, cinnamon powder or the like may be used as the flavoring agent. Of course, if necessary, these tablets and granule may be coated appropriately with sugar coating, gelatin coating or else.

Solid dosage forms for oral administration can include chewing gum, capsules, tablets, sublingual tablets, powders, granules, and gels. In such solid dosage forms, the active compound can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings, preferably film coatings.

To make tablets, the compounds can be admixed with pharmaceutically acceptable carriers known in the art such as, for example, vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like.

Sublingual administration refers to the administration in the mouth (e.g., under the tongue, between the cheek and gum, between the tongue and roof of the mouth). The highly vascular mucosal lining in the mouth is a convenient location for the compounds to be administered into the body.

In other embodiments, the solid dosage form can be packaged as granules or a powder in a pharmaceutically acceptable carrier, where the granules or powder are removed from the packaging and sprinkled on food or mixed with a liquid, such as water or juice, or where the granules are inserted into capsules. In this embodiment, the compounds described herein can be mixed with flavoring or sweetening agents. The packaging material can be plastic, coated paper, or any material that prevents water or moisture from reaching the granules and/or powder.

Liquid dosage forms for oral administration can include pharmaceutically acceptable emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the compounds can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents).

Each of the patents, patent applications, and publications cited herein are incorporated by reference herein in their entirety.

It will be apparent to one skilled in the art that various modifications can be made to the invention without departing from the spirit or scope of the appended claims.

What is claimed is:

1. A pharmaceutical composition comprising:
(A) an AMPA receptor antagonist, a pharmaceutically acceptable salt thereof, a hydrate thereof, or a hydrate of a pharmaceutically acceptable salt thereof;
(B) a cholinesterase inhibitor or a pharmaceutically acceptable salt thereof; an anti-neuropathic pain agent; or a mixture or combination thereof; and
(C) one or more pharmaceutically acceptable carriers.

2. The pharmaceutical composition of claim 1, wherein the AMPA receptor antagonist, pharmaceutically acceptable salt thereof, hydrate thereof, or hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is: 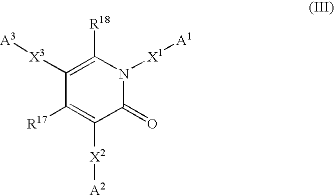
wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

3. The pharmaceutical composition of claim 1, wherein the AMPA receptor antagonist, a pharmaceutically acceptable salt thereof, a hydrate thereof, or a hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

4. The pharmaceutical composition of claim 1, wherein the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof; and galanthamine or a pharmaceutically acceptable salt thereof.

5. The pharmaceutical composition of claim 1, wherein the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818.

6. The pharmaceutical composition of claim 1, wherein the composition is used for treating neuropathic pain.

7. A combination comprising:
(A) an AMPA receptor antagonist, a pharmaceutically acceptable salt thereof, a hydrate thereof, or a hydrate of a pharmaceutically acceptable salt thereof; and
(B) a cholinesterase inhibitor or a pharmaceutically acceptable salt thereof; an anti-neuropathic pain agent; or a mixture or combination thereof.

8. The combination of claim 7, wherein the AMPA receptor antagonist, pharmaceutically acceptable salt thereof, hydrate thereof, or hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is: 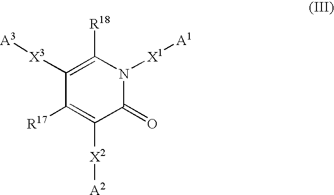
wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

9. The combination of claim 7, wherein the AMPA receptor antagonist, a pharmaceutically acceptable salt thereof, a hydrate thereof, or a hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

10. The combination of claim 7, wherein the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof; and galanthamine or a pharmaceutically acceptable salt thereof.

11. The combination of claim 7, wherein the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818.

12. The combination of claim 7, wherein (A) and (B) are administered separately to a patient or are administered to a patient in the form of a pharmaceutical composition.

13. The combination of claim 7, wherein the combination is used for treating neuropathic pain.

14. Use of compounds (A) and (B) for producing a pharmaceutical composition in the treatment of neuropathic pain, wherein (A) and (B) are:
(A) an AMPA receptor antagonist, a pharmaceutically acceptable salt thereof, a hydrate thereof, or a hydrate of a pharmaceutically acceptable salt thereof; and
(B) a cholinesterase inhibitor or a pharmaceutically acceptable salt thereof; an anti-neuropathic pain agent; or a mixture or combination thereof.

15. The use of claim 14, wherein the AMPA receptor antagonist, pharmaceutically acceptable salt thereof, hydrate thereof, or hydrate of the pharmaceutically acceptable salt thereof is a compound of Formula (III), a pharmaceutically acceptable salt thereof, a hydrate thereof, or a hydrate of a pharmaceutically acceptable salt thereof, wherein the compound of Formula (III) is: 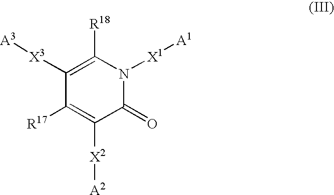
wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)_{N}—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

16. The use of claim 14, wherein the AMPA receptor antagonist, a pharmaceutically acceptable salt thereof, a hydrate thereof, or a hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

17. The use of claim 14, wherein the cholinesterase inhibitor or a pharmaceutically acceptable salt thereof is at least one compound selected from the group consisting of donepezil or a pharmaceutically acceptable salt thereof; rivastigmine or a pharmaceutically acceptable salt thereof; and galanthamine or a pharmaceutically acceptable salt thereof.

18. The use of claim 14, wherein the anti-neuropathic pain agent is at least one compound selected from the group consisting of gabapentin, pregabalin, duloxetine, mexiletine, lidocaine, amytriptyline, imipramine, clomipramine, desipramine, nortriptyline, maprotiline, paroxetine, fluoxetine, citalopram, venlafaxine, bupropion, carbamazepine, oxcabazepine, phenyloin, lamotrigine, valproate, topiramate, levetiracetam, tiagabine, lacosamide, brivaracetam tramadol, oxycodone, morphine, fentanyl, methadone, dextromethorphan, memantine, ketamine, riluzole, dronabinol, THC, capsaicin, clonidine, baclofen, tizanidine, AVP-923, desvenlafaxine succinate, bicifadine, OPC-14523, NGX4010, ralfinamide, XP-13512, KDS-2000, CNS-5161, V-3381, GW-493838, GW-353162, CCI-1008, SS-RBX, cannabidiol, RGH-896, SAB-378, TV-1901, ABT-894, TAK-583 and AGN-203818.

19. The use of claim 14, wherein (A) and (B) are administered separately to a patient or are administered to a patient in the form of a pharmaceutical composition.

20. Compounds (A) and (B) for use in the treatment of neuropathic pain, wherein (A) and (B) are:
(A) an AMPA receptor antagonist, a pharmaceutically acceptable salt thereof, a hydrate thereof, or a hydrate of a pharmaceutically acceptable salt thereof, and
(B) a cholinesterase inhibitor or a pharmaceutically acceptable salt thereof; an anti-neuropathic pain agent; or a mixture or combination thereof.

21. A kit comprising the pharmaceutical composition of any one of claims 1 to 6 or the combination of any one of claims 7 to 13.

22. A method for treating neuropathic pain comprising administering to a patient in need thereof a therapeutically effective amount of the pharmaceutical composition of any one of claims 1 to 6 or a therapeutically effective amount of the combination of any one of claims 7 to 13.
